(19) United States
(12) Patent Application Publication
(10) Pub. No.: US 2025/0241965 A1
(43) Pub. Date: Jul. 31, 2025

(54) METHOD TO IMPROVE CELL VIABILITY OF CARDIOMYOCYTES

(71) Applicant: UNIVERSITY OF WASHINGTON, Seattle, WA (US)

(72) Inventors: Steven KATTMAN, Seattle, WA (US); Jennifer POTTER, Seattle, WA (US); Charles E. MURRY, Seattle, WA (US)

(73) Assignee: UNIVERSITY OF WASHINGTON, Seattle, WA (US)

(21) Appl. No.: 18/854,233

(22) PCT Filed: Apr. 4, 2023
(86) PCT No.: PCT/US2023/017396

§ 371 Date: Oct. 4, 2024

Related U.S. Application Data
(60) Provisional application No. 63/327,650, filed on Apr. 5, 2022.
(60) Provisional application No. 63/413,471, filed on Oct. 5, 2022.

(51) Int. Cl. A61K 35/34; C12N 5/077

(52) U.S. Cl. CPC A61K 35/34; C12N 5/0657; C12N 2501/999; C12N 2506/45

(57) ABSTRACT

The methods and compositions relate to the improvement of cell viability regarding in vitro differentiated cardiomyocytes. Addition of ROCK-inhibitors to pluripotent stem cells in combination with factors modulating differentiation pathways improves in-vitro differentiated cardiomyocyte viability and fitness.

### CROSS REFERENCE TO RELATED APPLICATIONS

This application claims benefit under 35 U.S.C. § 119 (e) to U.S. Provisional Application No. 63/327,650 filed Apr. 5, 2022, and U.S. Provisional Application No. 63/413,471 filed Oct. 5, 2022, the contents each of which are incorporated herein by reference in their entireties.

### TECHNICAL FIELD

The technology described herein relates to in vitro differentiated cardiomyocytes and compositions and methods that use them.

### BACKGROUND

Methods involving transplant of cardiomyocytes into sites of cardiac damage can benefit from improvements in growth and survival of in vitro-differentiated cardiomyocytes.

### SUMMARY

The methods and compositions provided herein are related to the discovery that a ROCK inhibitor, such as Y-27632, improves cell viability during the in vitro differentiation of cardiomyocytes. Improved survival of cardiomyocytes helps to improve cell and tissue engineering efforts.

In one aspect, provided herein is an in vitro differentiation method for preparing a cardiomyocyte from a stem cell, the method comprising contacting the stem cell with an inhibitor of Rho-associated, coiled-coil containing protein kinase (ROCK) throughout differentiation from stem cell to cardiomyocyte.

In one embodiment of this and any other aspect, the ROCK inhibitor is Y-27632.

In another embodiment of this and any other aspect, the ROCK inhibitor throughout differentiation promotes cell viability relative to differentiation in which ROCK inhibitor contacting is not throughout differentiation.

In another embodiment of this and any other aspect, the stem cell is a pluripotent stem cell or a cardiac progenitor cell.

In another embodiment of this and any other aspect, the stem cell is human.

In another embodiment of this and any other aspect, the stem cell is an induced pluripotent stem cell.

In another embodiment of this and any other aspect, the stem cell is an embryonic stem cell.

In another embodiment of this and any other aspect, the stem cell is not an embryonic stem cell.

In another embodiment of this and any other aspect, the method further comprises contacting the stem cell or a cell differentiating from the stem cell with a small molecule selected from CHIR99021 and WIKI4.

In another embodiment of this and any other aspect, the method further comprises contacting the stem cell or a cell differentiating from the stem cell with CHIR99021 and WIKI4.

In another aspect, provided herein is a composition comprising a stem cell, a ROCK inhibitor, and one or both of CHIR99021 and WIKI4.

In one embodiment of this and any other aspect, the ROCK inhibitor is Y-27632.

In another embodiment of this and any other aspect, the stem cell is a pluripotent stem cell or a cardiac progenitor cell.

In another embodiment of this and any other aspect, the stem cell is human.

In another embodiment of this and any other aspect, the stem cell is an induced pluripotent stem cell.

In another embodiment of this and any other aspect, the stem cell is an embryonic stem cell.

In another embodiment of this and any other aspect, the stem cell is not an embryonic stem cell.

In another aspect, provided herein is a transplant composition comprising a cardiomyocyte produced by the methods described herein.

In one embodiment of this and any other aspect, the transplant composition further comprises a pharmaceutically acceptable carrier.

In another embodiment of this and any other aspect, the transplant composition further comprises a gel, scaffold or matrix.

In another embodiment of this and any other aspect, the gel, scaffold or matrix is biodegradable.

In another embodiment of this and any other aspect, the transplant composition further comprises any one or more of a solubilized basement membrane protein or preparation thereof, an immunosuppressive agent, a pan-caspase inhibitor, an anti-apoptotic agent, IGF-1, and a KATP channel opening agent.

In another aspect, provided herein is pharmaceutical composition comprising a cardiomyocyte produced by a method as described herein, in combination with a pharmaceutically acceptable carrier.

In another aspect, provided herein is a pharmaceutical composition comprising a transplant composition as described herein, in combination with a pharmaceutically acceptable carrier.

In another aspect, described herein is a method for improving cardiac function in a subject in need thereof, the method comprising administering a cardiomyocyte produced by a method as described herein, or a transplant composition or pharmaceutical composition as described herein to cardiac tissue of the subject in need thereof.

In one embodiment of this and any other aspect, the subject is human.

In another embodiment of this and any other aspect, the subject has damaged cardiac tissue resulting from acute or chronic injury.

In another embodiment of this and any other aspect, provided herein is a cardiomyocyte produced by a method as described herein, or a composition as described herein, for use in a method for improving cardiac function in a subject in need thereof, said method comprising administering a cardiomyocyte produced by as described herein, or a composition as described herein to cardiac tissue of the subject in need thereof.

In one embodiment of this and any other aspect, the subject is human.

In one embodiment of this and any other aspect, the subject has damaged cardiac tissue resulting from acute or chronic injury.

### BRIEF DESCRIPTION OF THE DRAWINGS

FIG. 1A depicts the timeline, base media, media supplements, small molecules for the differentiation of pluripotent stem cells to cardiomyocytes. Addition of CHIR, Wnt agonist, occurs at Day 3 of differentiation, while the Wnt antagonist Wiki, is added either 48 or 44 hours after CHIR addition. The timing from CHIR to WIKI addition is optimized for each cell line and/or reagent change to most efficiently induce cardiomyocye differentiation.

FIG. 1B examines the difference between adding additional WIKI4 on day 6 of the differentiation vs a media change with no additional WIKI4 on day 6. Both conditions induced efficient differentiation to mesoderm, indicated by expression of CD56 and PDGFRa analyzed by flow cytometry. When allowed to continue to differentiate using conditions depicted in FIG. 1A, no additional WIKI on day 6 conditions resulted in efficient differentiation to cardiomyocytes by Day 20 of differentiation, measured by intracellular flow cytometry for the expression of cardiac tropoinin T (CTNT). Few EPCAM+cells were detected demonstrating lack of epithelial type cells in the culture. Additionally, no additional WIKI resulted in roughly 8E5 cells/mL in the final culture. In contrast, conditions of additional WIKI resulted in extensive loss of cells and too few cells to measure either cell counts of CTNT expression.

FIG. 2 (left) shows the concentration of cells over time after the addition or removal of small molecules and/or media in cell cultures differentiated in 8 uM CHIR, no additional WIKI (A) or 8 uM CHIR with additional WIKI (B). Arrows indicate relevant process steps for orientation. FIG. 2 right, indicates the possible mechanisms for cell loss after day 7 in conditions A or B.

FIG. 3 (top) illustrates the current best practice (CBP) differentiation methodology including timeline, base media, media supplements and the addition of small molecules. FIG. 3 (bottom) the experimental set up to test three different methodologies designed to improve the cell yield in comparison to CBP (1). To improve the total cell yield, adding an additional media change on the day just prior to the initiation of significant cell loss (2), or the addition of ROCK inhibitor at the points of small molecule addition (CHIR or WIKI, respectively) were tested. To monitor for the fate of cells in the experiment, the cultures were monitored for cell counts (both in single cell suspension and in aggregates), glucose, lactate, and pH using a Blood Gas Analyzer (BGA); apoptosis was analyzed by AnnexinV staining; additionally, mesoderm formation was monitored by flow cytometry for CD56 and PDGFRa to determine if the addition of ROCK inhibitor changed the developmental trajectory of the cells.

FIG. 4 (top) once again illustrates the experimental design as described in FIG. 4, for reference. FIG. 4 (bottom) indicates the cell counts at multiple time-points from each condition during the differentiation of experiment #19. 19-1 is CBP conditions, 19-2 is media change at day 6 conditions (including the addition of Wiki 5 uM), 19-3 is conditions that received ROCK inhibitor at the time of small molecule addition. At day 6 of differentiation cells numbers in condition 19-3 were elevated to the point of needing to double the media volume and therefore cells were split into 19-3A and 19-3B. As illustrated in the figure, CBP conditions led to cell loss, as anticipated, media change at day 6 resulted in even more rapid loss of cells, while the addition of ROCK inhibitor not only rescued cells/mL, but resulted in significantly higher cell numbers and the need to double the media volume.

FIG. 5 shows testing if the cell loss in the CBP conditions was due to cells sluffing off the aggregates and being in single cell suspension, as opposed to the aggregate, both cells in aggregates (A) and single cell suspension (B) were counted during the differentiation in CBP conditions. As there wasn't an accumulation of cells in single cell suspension during the differentiation, cells sluffing off the aggregates could not account for the cell loss during CBP conditions.

FIG. 6 illustrates that apoptosis correlates with the cell loss detected in CBP conditions (19-1) and media change (including Wiki) conditions (19-2), in comparison to ROCK inhibitor conditions (19-3). This illustration also includes an additional experimental set which includes addition of ROCK inhibitor at time-points of small molecule addition (20-1), or CBP conditions (20-2). As seen in the lower right panel, conditions of cell loss displayed elevated AnnexinV staining indicating apoptosis, in comparison to conditions where cell numbers were rescued under ROCK conditions. Flow cytometry dot plot panels indicate three examples of low (19-3), medium (20-2), and high (19-2) AnnexinV staining.

FIG. 7 depicts that cardiomyocyte purity was similar in each condition tested, despite the significantly different cell yield from each experimental condition. Using ES cells as a control, there is very little epithelial cell commitment, indicated by flow cytometry stains for EPCAM and CD56. Additionally, cardiomyocyte purity, indicated by intracellular staining for the cardiac isoform of troponin T (CTNT) and flow cytometry, indicates that cardiomyocyte purity was >90% for each condition tested. At the end of culture, all samples which made it to the end of differentiation from experiment 19 (19-1, 19-3A, 19-3B) were pooled, resulting in roughly 1.2 billion cell yield.

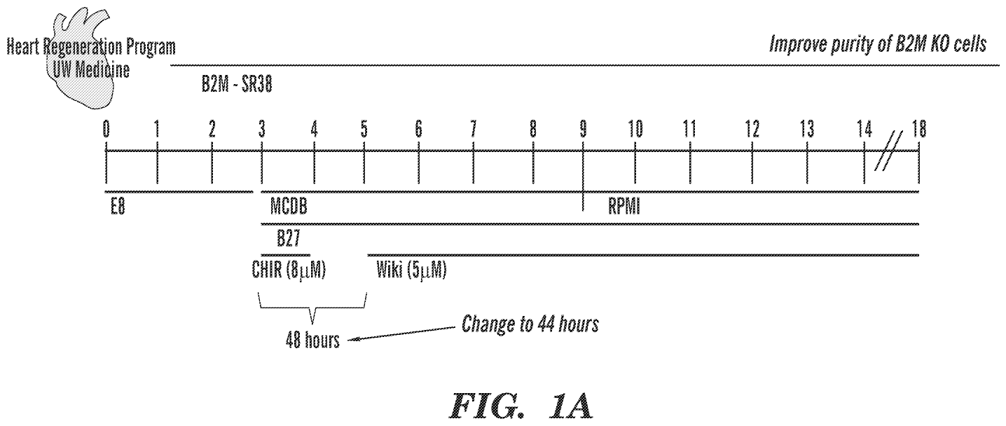

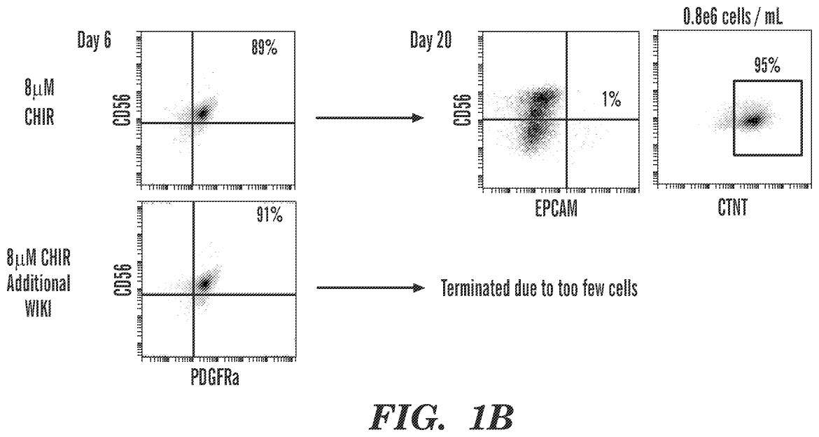

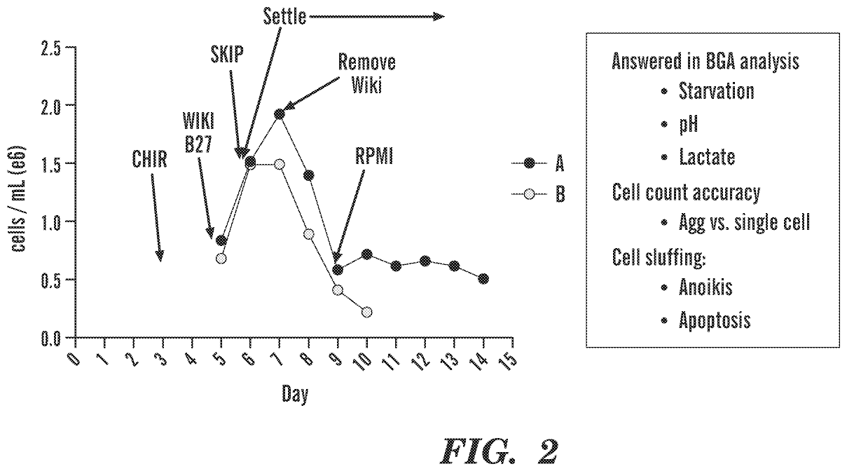

### DETAILED DESCRIPTION

### Definitions

The term “differentiate” or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g., a human cardiac progenitor cell or mid-primitive streak cardiogenic mesoderm progenitor cell), which in turn can differentiate into other types of precursor cells further down the pathway (such as a tissue specific precursor, such as a cardiomyocyte progenitor cell), and then to an end-stage differentiated cell (e.g., a cardiomyocyte), which plays a characteristic role in a certain tissue type, and may or may not retain the capacity to proliferate further.

The term “pluripotent” or “pluripotent stem cell (PSC)” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

As used herein, the terms “induced pluripotent stem cell,” “iPSC,” “hPSC,” and “human pluripotent stem cell” are used interchangeably herein and refer to a pluripotent cell artificially derived from a differentiated somatic cell (e.g., by reprogramming using one or more methods known in the art). iPSCs are capable of self-renewal and differentiation into cell fate-committed stem cells, including cells of the cardiac lineages, as well as various types of mature cells.

As used herein, “in vitro-differentiated cardiomyocytes” refers to cardiomyocytes that are generated in culture, typically, but not necessarily via step-wise differentiation from a precursor cell such as a human embryonic stem cell, an induced pluripotent stem cell, an early mesoderm cell, a lateral plate mesoderm cell or a cardiac progenitor cell. Thus, while cardiomyocytes in vivo are ultimately derived from a stem cell, i.e., during development of a tissue or organism, a stem cell-derived cardiomyocyte as described herein has been created by in vitro differentiation from a stem cell. Methods for differentiating stem cells in vitro to cardiomyocytes are known in the art and described elsewhere herein. In one embodiment, the cardiomyocytes are differentiated from pluripotent stem cells (e.g., PSC-CMs).

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of cardiomyocytes, refers to a population of cells that contains fewer than about 20%, more preferably fewer than about 15%, 10%, 8%, 7%, most preferably fewer than about 5%, 4%, 3%, 2%, 1%, or less than 1%, of cells that are not cardiomyocytes, respectively.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell. Similarly, “PSC-CMs” or “pluripotent stem cell-derived cardiomyocytes” are used interchangeably to refer to cardiomyocytes derived from a pluripotent stem cell. In some embodiments, the terms “hPSC-CM” or “human pluripotent stem cell derived cardiomyocytes” are used interchangeably to refer to cardiomyocytes derived from a human pluripotent stem cell.

As used herein, the term “isoform selective” refers to the property of an agent, e.g., an inhibitor or activator of a factor, in which the agent inhibits or activates one isoform of the factor but does not substantially inhibit or activate one or more different isoforms of the factor. In this context, “does not substantially inhibit or activate” means that the isoform selective inhibitor or activator is at least 100× more active (whether inhibitory or stimulatory) against a target or reference isoform than against other isoforms of the factor.

As used herein, the terms “transplanting,” “administering” or “engraftment” are used in the context of the placement of cells, e.g., cardiomyocytes produced as described herein, into a subject, by a method or route which results in at least partial localization of the introduced cells at a desired site, such as a site of injury or repair, such that a desired effect(s) is produced. The cells e.g., cardiomyocytes, can be implanted directly to the heart or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of cardiomyocytes is desired as cardiomyocytes do not proliferate to an extent that the heart can heal from an acute injury comprising cell death. Thus, a graft can be used to replace lost cells that occur during injury. A graft can also be used to provide support to cardiac tissue during recovery from a cardiac injury. Methods for improving engraftment or preventing engraftment arrhythmias, such as those described in e.g., US 2020-0085880, WO 2020/190739, or WO 2021/163037, the contents of each of which are incorporated herein by reference in their entirety, can be combined with the methods and compositions described herein. In one embodiment, engraftment is used to refer to cardiomyocytes that have formed functional gap junctions with endogenous cardiomyocytes in the subject.

As used herein, the term “contacting” when used in reference to a cell, encompasses introducing a cell, agent, surface, scaffold etc. to the cell in a manner that permits physical contact of the cell with the cell, agent, surface, scaffold etc.

As used herein, the term, “cardiac disease” refers to a disease that affects the cardiac tissue of a subject. Non-limiting examples of cardiac diseases include cardiomyopathy, cardiac arrhythmias, myocardial infarction, heart failure, cardiac hypertrophy, long QT syndrome, arrhythmogenic right ventricular dysplasia (ARVD), catecholaminergic polymorphic ventricular tachycardia (CPVT), Barth syndrome, congenital defects, and Duchenne muscular dystrophy.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease. A subject can be of any age including, e.g., a fetus, a neonate, a toddler, a child, an adolescent, an adult, a geriatric subject etc.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to measurements, “about” means+/−5%.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It will be appreciated that, although specific embodiments of the invention have been described herein for purposes of illustration, various modifications may be made without deviating from the spirit and scope of the invention. Accordingly, the invention is not limited except as by the claims.

### Cardiovascular Diseases

The methods described herein can be used to treat, ameliorate, prevent or slow the progression of a number of diseases or their symptoms, such as those resulting in pathological damage to the structure and/or function of the heart (e.g., remodeling in response to heart failure).

A cardiovascular disease is a disease that affects the heart and/or circulatory system of a subject. Such cardiac diseases or cardiac-related disease include, but are not limited to, myocardial infarction, cardiac arrhythmia, heart failure, atherosclerotic heart disease, cardiomyopathy, congenital heart defect (e.g., non-compaction cardiomyopathy, septal defects, hypoplastic left heart), hypertrophic cardiomyopathy, dilated cardiomyopathy, cardiac hypertrophy, myocarditis, arrhythmogenic right ventricular dysplasia (ARVD), long QT syndrome, catecholaminergic polymorphic ventricular tachycardia (CPVT), Barth syndrome, valvular stenosis, regurgitation, ischemia, fibrillation, polymorphic ventricular tachycardia, and muscular dystrophies such as Duchenne or related cardiac disease, and cardiomegaly. Generally, the methods and compositions described herein will be most beneficial for the treatment of cardiac diseases or disorders with impaired contractility, for example, heart failure, myocardial infarction, and cardiomyopathies.

Symptoms of cardiovascular disease can include but are not limited to syncope, fatigue, shortness of breath, chest pain, lower limb edema, and palpitations. A cardiovascular disease is generally diagnosed by a physical examination, blood tests, and/or an electrocardiogram (EKG). An abnormal EKG is an indication that the subject has an abnormal cardiac rhythm or cardiac arrhythmia.

In some embodiments of any of the aspects, the subject has or is at risk for having a cardiovascular disease, cardiac damage or a cardiac event.

### Pluripotent Stem Cell Sources

The methods and compositions described herein can be used to generate cardiomyocytes by in vitro differentiation from e.g., embryonic stem cells, pluripotent stem cells, such as induced pluripotent stem cells, or other stem cells that permit such differentiation and are modified as described herein. The following describes various stem cells that can be used to prepare cardiomyocytes.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of pluripotent stem cells can include amnion-derived or placental-derived stem cells. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Cardiomyocytes useful in the methods and compositions described herein can be differentiated from both embryonic stem cells and induced pluripotent stem cells, among others. In one embodiment, the compositions and methods provided herein use human cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells and methods for their retrieval are well known in the art and are not described in detail herein. A cell has the phenotype of an embryonic stem cell if it possesses one or more of the unique characteristics of an embryonic stem cell such that that cell can be distinguished from other cells. Exemplary distinguishing embryonic stem cell characteristics include, without limitation, morphology, gene expression or marker profile, proliferative capacity, differentiation capacity, karyotype, responsiveness to particular culture conditions, and the like.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin.

Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns.

In some embodiments, the methods and compositions described herein utilize cardiomyocytes that are differentiated in vitro from induced pluripotent stem cells. An advantage of using iPSCs to generate cardiomyocyte for the compositions described herein is that the cells can be derived from the same subject to which the desired human cardiomyocytes are to be administered. That is, a somatic cell can be obtained from a subject, reprogrammed to an induced pluripotent stem cell, and then re-differentiated into a human cardiomyocyte cell to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects.

In some embodiments, the cardiomyocytes useful for the compositions described herein are derived from non-autologous or allogeneic sources.

In some embodiments, an iPSC is a cell that has been reprogrammed, a process that alters or reverses the differentiation state of a differentiated cell (e.g., a somatic cell). Stated another way, reprogramming is a process of driving the differentiation of a cell backwards to a more undifferentiated or more primitive type of cell. Reprogramming of somatic cells to induced pluripotent stem cells is known in the art and is not described in detail herein. iPS cells can be generated or derived from terminally differentiated somatic cells, as well as from adult stem cells, or somatic stem cells. That is, a non-pluripotent progenitor cell can be rendered pluripotent or multipotent by reprogramming.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

Methods of in vitro differentiating stem cells to cardiomyocytes are known in the art and/or described herein below.

### ROCK Inhibitors

In some embodiments of the technology described herein, a stem cell is contacted with at least one ROCK inhibitor during differentiation of the stem cell to a cardiomyocyte.

The small GTP-binding proteins of the Rho family are involved in the regulation of various aspects of cell motility, shape, proliferation and apoptosis. Rho kinases, also referred to as ROCKs, are serine/threonine kinases activated by GTP-bound Rho proteins that phosphorylate downstream targets in the ROCK pathway. Phosphorylation targets include, but are not limited to myosin light chain phosphatase, LIM kinases, adducin, and ezrin-radixon-moesin (ERM) proteins. Other or related Rho kinase functions include, for example, regulation of smooth muscle cell contraction, cell migration, and maintenance of cell viability and morphology, in part by regulating stress fibers and focal adhesions. A ROCK inhibitor is a compound that targets a ROCK and inhibits ROCK-mediated signal transduction. Examples of ROCK inhibitors specifically contemplated for use in the methods and compositions described herein include, but are not limited to those in Table 1 as shown below. In some embodiments, the ROCK inhibitor is Y-27632. Additional ROCK inhibitors that can be used, alone or, for example, together with Y-27632, include those described in Liao et al. 2007. J Cardiovasc Pharmacol. 50 (1): 17-24.

TABLE 1
Examples of ROCK inhibitors:
Isoquinoline and pyridine/pyrrolopyridine based ROCK inhibitors

| Name | Structure |
|---|---|
| Fasudil | 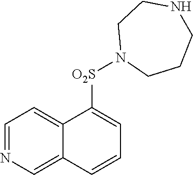 [SMILES: O=S(=O)(c1cccc2cnccc12)N1CCCNCC1] |
| Hydroxyfasudil | 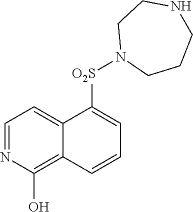 [SMILES: O=S(=O)(c1cccc2c(O)nccc12)N1CCCNCC1] |
| H-1152P | 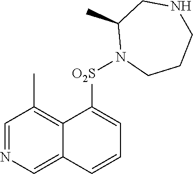 [SMILES: Cc1cncc2cccc(S(=O)(=O)N3CCCNC[C@@H]3C)c12] |
| Y-27362 | 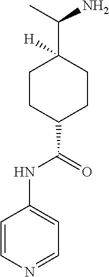 |
| Y-30141 | 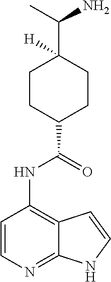 |
| Y-32885 | 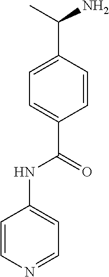 [SMILES: C[C@@H](N)c1ccc(C(=O)Nc2ccncc2)cc1] |
| Y-39983 | 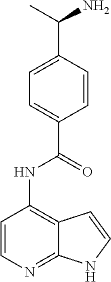 [SMILES: C[C@@H](N)c1ccc(C(=O)Nc2ccnc3[nH]ccc23)cc1] |

Indazole based ROCK inhibitors

| | |
|---|---|
| Kirin-indazole based ROCK inhibitors | 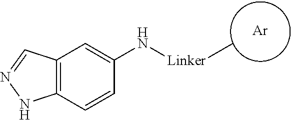 |
| Kirin-indazole based ROCK-II inhibitor | 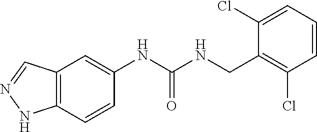 [SMILES: O=C(NCc1c(Cl)cccc1Cl)Nc1ccc2[nH]ncc2c1] |
| Kirin-indazole based ROCK-II inhibitor | 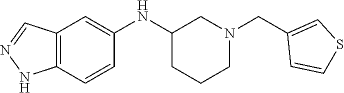 [SMILES: c1cc(CN2CCCC(Nc3ccc4[nH]ncc4c3)C2)cs1] |
| Kirin-indazole based ROCK-II inhibitor | 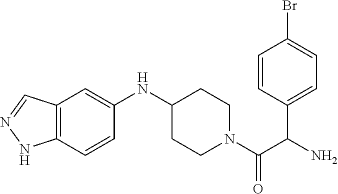 [SMILES: NC(C(=O)N1CCC(Nc2ccc3[nH]ncc3c2)CC1)c1ccc(Br)cc1] |
| Kirin-indazole based ROCK-II inhibitor | 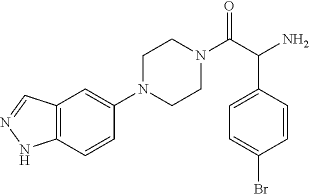 [SMILES: NC(C(=O)N1CCN(c2ccc3[nH]ncc3c2)CC1)c1ccc(Br)cc1] |
| Kirin-indazole based ROCK-II inhibitor | 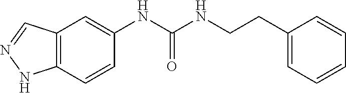 [SMILES: O=C(NCCc1ccccc1)Nc1ccc2[nH]ncc2c1] |
| Kirin-indazole based ROCK-I inhibitor | 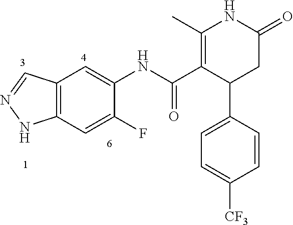 [SMILES: CC1=C(C(=O)Nc2cc3cn[nH]c3cc2F)C(c2ccc(C(F)(F)F)cc2)CC(=O)N1] |
| DW1865 | 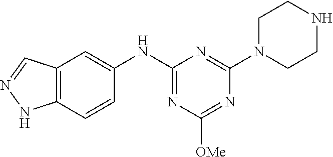 [SMILES: COc1nc(Nc2ccc3[nH]ncc3c2)nc(N2CCNCC2)n1] |
| KD025 | 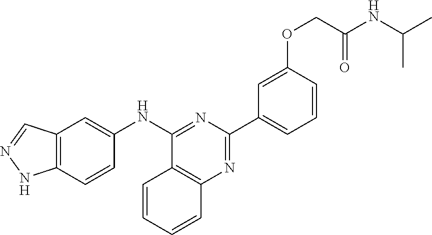 [SMILES: CC(C)NC(=O)COc1cccc(-c2nc(Nc3ccc4[nH]ncc4c3)c3ccccc3n2)c1] |
| Kirin-indazole based ROCK-I inhibitor | 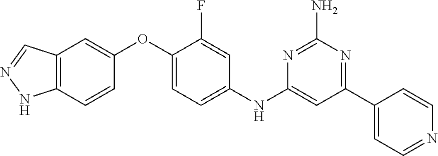 [SMILES: Nc1nc(Nc2ccc(Oc3ccc4[nH]ncc4c3)c(F)c2)cc(-c2ccncc2)n1] |
| Kirin-indazole based ROCK-I inhibitor | 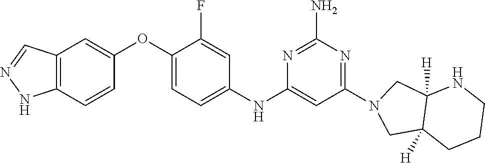 [SMILES: Nc1nc(Nc2ccc(Oc3ccc4[nH]ncc4c3)c(F)c2)cc(N2C[C@@H]3CCCN[C@@H]3C2)n1] |
| Kirin-indazole based ROCK-II inhibitor | 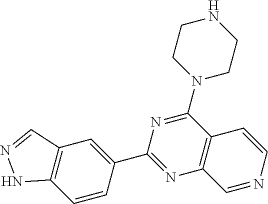 [SMILES: c1cc2c(N3CCNCC3)nc(-c3ccc4[nH]ncc4c3)nc2cn1] |
| Kirin-indazole based ROCK inhibitor | 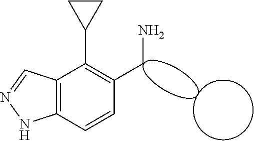 [SMILES: NCc1ccc2[nH]ncc2c1C1CC1] |

Pyridine, pyrrolopyridine, and pyrimidine based ROCK inhibitors

| | |
|---|---|
| Pyridine, pyrrolopyridine, and pyrimidine based ROCK inhibitor | 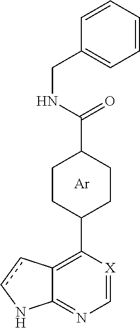 |
| Pyridine, pyrrolopyridine, and pyrimidine based ROCK inhibitor | 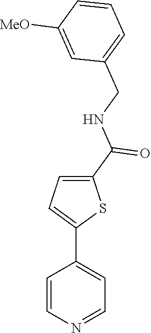 [SMILES: COc1cccc(CNC(=O)c2ccc(-c3ccncc3)s2)c1] |
| Pyridine, pyrrolopyridine, and pyrimidine based ROCK inhibitor | 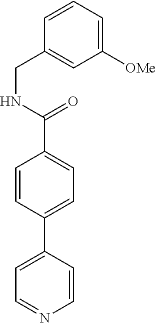 [SMILES: COc1cccc(CNC(=O)c2ccc(-c3ccncc3)cc2)c1] |
| Pyridine, pyrrolopyridine, and pyrimidine based ROCK inhibitor | 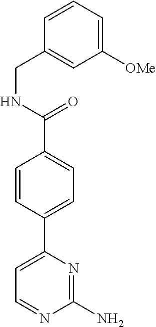 [SMILES: COc1cccc(CNC(=O)c2ccc(-c3ccnc(N)n3)cc2)c1] |
| Pyridine, pyrrolopyridine, and pyrimidine based ROCK inhibitor | 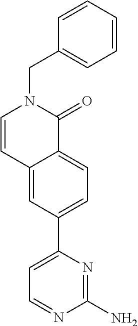 [SMILES: Nc1nccc(-c2ccc3c(=O)n(Cc4ccccc4)ccc3c2)n1] |
| Pyridine, pyrrolopyridine, and pyrimidine based ROCK-II inhibitor | 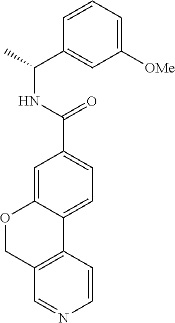 [SMILES: COc1cccc([C@@H](C)NC(=O)c2ccc3c(c2)OCc2cnccc2-3)c1] |
| Pyridine, pyrrolopyridine, and pyrimidine based ROCK inhibitor | 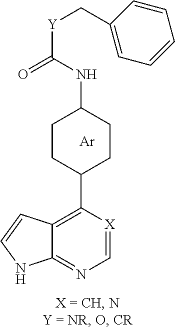 |
| Pyridine, pyrrolopyridine, and pyrimidine based ROCK-II inhibitor | 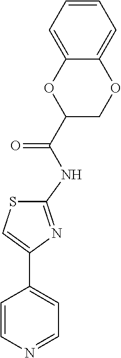 [SMILES: O=C(Nc1nc(-c2ccncc2)cs1)C1COc2ccccc2O1] |
| Pyridine, pyrrolopyridine, and pyrimidine based ROCK-I inhibitor | 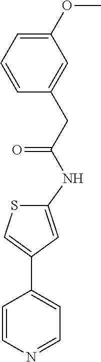 [SMILES: COc1cccc(CC(=O)Nc2cc(-c3ccncc3)cs2)c1] |
| Pyridine, pyrrolopyridine, and pyrimidine based ROCK-I inhibitor | 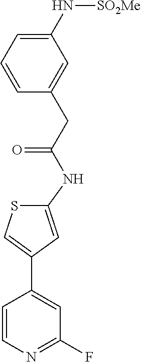 [SMILES: CS(=O)(=O)Nc1cccc(CC(=O)Nc2cc(-c3ccnc(F)c3)cs2)c1] |
| Pyridine, pyrrolopyridine, and pyrimidine based ROCK-I inhibitor | 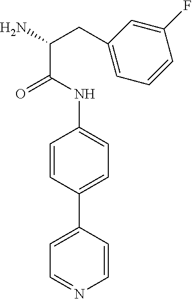 [SMILES: N[C@H](Cc1cccc(F)c1)C(=O)Nc1ccc(-c2ccncc2)cc1] |
| AS1892802 | 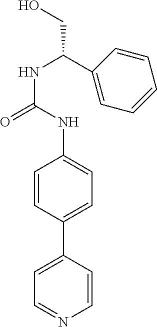 [SMILES: O=C(Nc1ccc(-c2ccncc2)cc1)N[C@H](CO)c1ccccc1] |
| Pyridine, pyrrolopyridine, and pyrimidine based ROCK-II inhibitor | 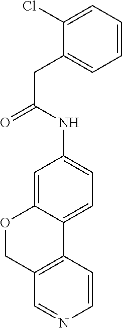 [SMILES: O=C(Cc1ccccc1Cl)Nc1ccc2c(c1)OCc1cnccc1-2] |
| Pyridine, pyrrolopyridine, and pyrimidine based ROCK-II inhibitor | 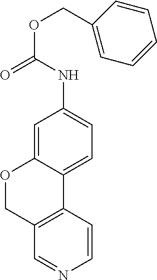 [SMILES: O=C(Nc1ccc2c(c1)OCc1cnccc1-2)OCc1ccccc1] |
| Pyridine, pyrrolopyridine, and pyrimidine based ROCK inhibitor | 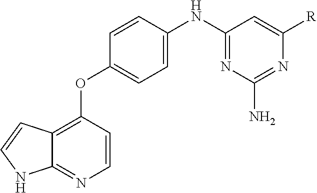 |
| Pyridine, pyrrolopyridine, and pyrimidine based ROCK inhibitor | 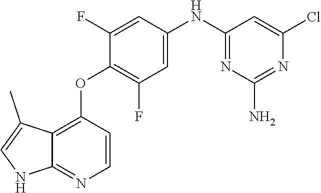 [SMILES: Cc1c[nH]c2nccc(Oc3c(F)cc(Nc4cc(Cl)nc(N)n4)cc3F)c12] |
| Pyridine, pyrrolopyridine, and pyrimidine based ROCK inhibitor | 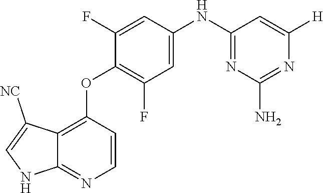 [SMILES: N#Cc1c[nH]c2nccc(Oc3c(F)cc(Nc4ccnc(N)n4)cc3F)c12] |
| Pyridine, pyrrolopyridine, and pyrimidine based ROCK inhibitor | 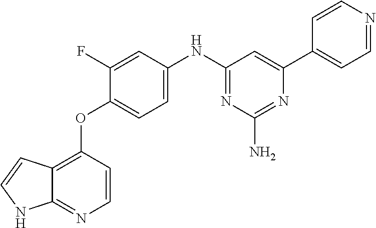 [SMILES: Nc1nc(Nc2ccc(Oc3ccnc4[nH]ccc34)c(F)c2)cc(-c2ccncc2)n1] |

Pyrazole-based ROCK Inhibitors

| | |
|---|---|
| Pyrazole-based ROCK Inhibitor | 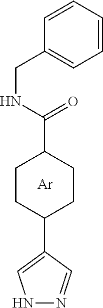 |
| Pyrazole-based ROCK Inhibitor | 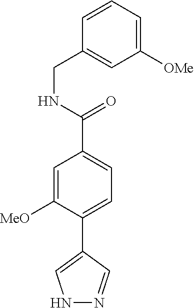 [SMILES: COc1cccc(CNC(=O)c2ccc(-c3cn[nH]c3)c(OC)c2)c1] |
| Pyrazole-based ROCK Inhibitor | 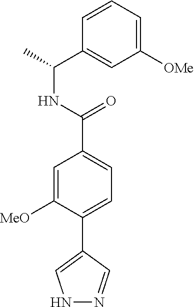 [SMILES: COc1cccc([C@@H](C)NC(=O)c2ccc(-c3cn[nH]c3)c(OC)c2)c1] |
| Pyrazole-based ROCK Inhibitor | 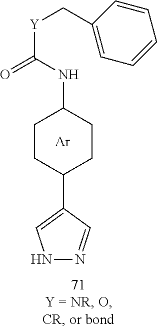 |
| Pyrazole-based ROCK Inhibitor | 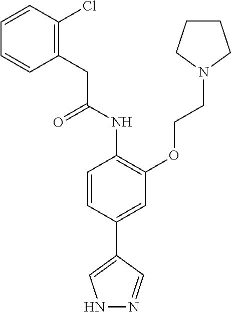 [SMILES: O=C(Cc1ccccc1Cl)Nc1ccc(-c2cn[nH]c2)cc1OCCN1CCCC1] |
| Pyrazole-based ROCK Inhibitor | 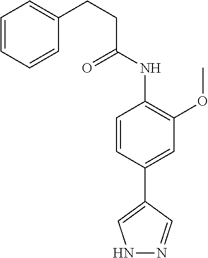 [SMILES: COc1cc(-c2cn[nH]c2)ccc1NC(=O)CCc1ccccc1] |
| SR3677 | 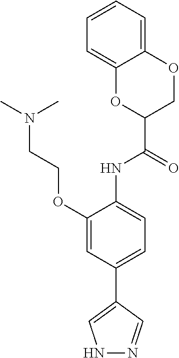 [SMILES: CN(C)CCOc1cc(-c2cn[nH]c2)ccc1NC(=O)C1COc2ccccc2O1] |
| SR3850 | 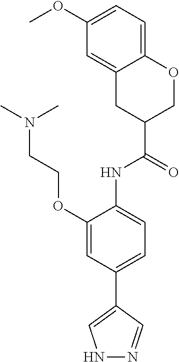 [SMILES: COc1ccc2c(c1)CC(C(=O)Nc1ccc(-c3cn[nH]c3)cc1OCCN(C)C)CO2] |
| Pyrazole-based ROCK Inhibitor | 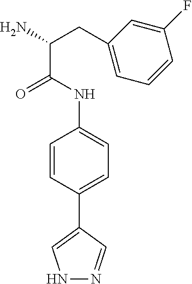 [SMILES: N[C@H](Cc1cccc(F)c1)C(=O)Nc1ccc(-c2cn[nH]c2)cc1] |
| Pyrazole-based ROCK Inhibitor | 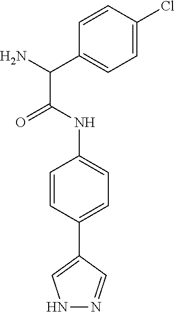 [SMILES: NC(C(=O)Nc1ccc(-c2cn[nH]c2)cc1)c1ccc(Cl)cc1] |
| SR7043 | 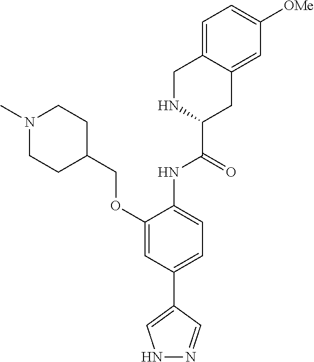 [SMILES: COc1ccc2c(c1)C[C@H](C(=O)Nc1ccc(-c3cn[nH]c3)cc1OCC1CCN(C)CC1)NC2] |
| Pyrazole-based ROCK Inhibitor | 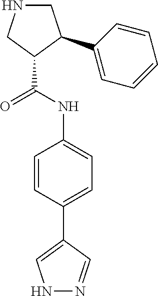 [SMILES: O=C(Nc1ccc(-c2cn[nH]c2)cc1)[C@@H]1CNC[C@H]1c1ccccc1] |
| Pyrazole-based ROCK Inhibitor | 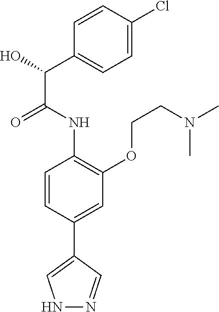 [SMILES: CN(C)CCOc1cc(-c2cn[nH]c2)ccc1NC(=O)[C@H](O)c1ccc(Cl)cc1] |
| SR6442 | 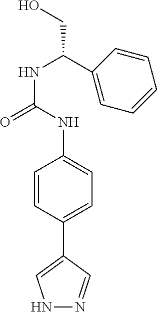 [SMILES: O=C(Nc1ccc(-c2cn[nH]c2)cc1)N[C@H](CO)c1ccccc1] |
| Pyrazole-based ROCK Inhibitor | 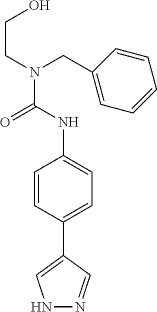 [SMILES: O=C(Nc1ccc(-c2cn[nH]c2)cc1)N(CCO)Cc1ccccc1] |
| SR5834 | 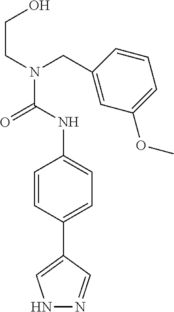 [SMILES: COc1cccc(CN(CCO)C(=O)Nc2ccc(-c3cn[nH]c3)cc2)c1] |
| Pyrazole-based ROCK Inhibitor | 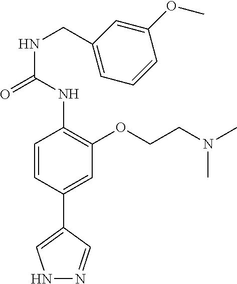 [SMILES: COc1cccc(CNC(=O)Nc2ccc(-c3cn[nH]c3)cc2OCCN(C)C)c1] |
| Pyrazole-based ROCK Inhibitor | 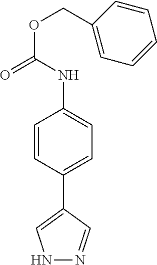 [SMILES: O=C(Nc1ccc(-c2cn[nH]c2)cc1)OCc1ccccc1] |

Methylenephenyl-substituted pyrazole based ROCK inhibitors

| | |
|---|---|
| Methylenephenyl-substituted pyrazole based ROCK inhibitor | 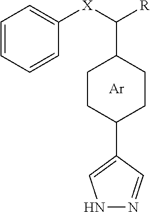 |
| Methylenephenyl-substituted pyrazole based ROCK-II inhibitor | 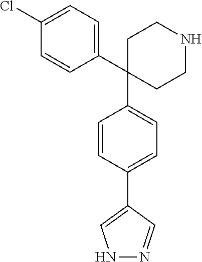 [SMILES: Clc1ccc(C2(c3ccc(-c4cn[nH]c4)cc3)CCNCC2)cc1] |
| Methylenephenyl-substituted pyrazole based ROCK-II inhibitor | 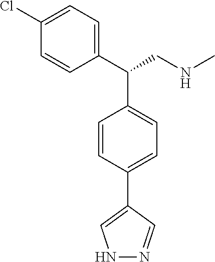 [SMILES: CNC[C@@H](c1ccc(Cl)cc1)c1ccc(-c2cn[nH]c2)cc1] |
| Methylenephenyl-substituted pyrazole based ROCK-II inhibitor | 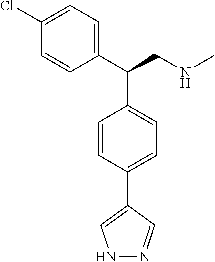 [SMILES: CNC[C@H](c1ccc(Cl)cc1)c1ccc(-c2cn[nH]c2)cc1] |
| Methylenephenyl-substituted pyrazole based ROCK-II inhibitor | 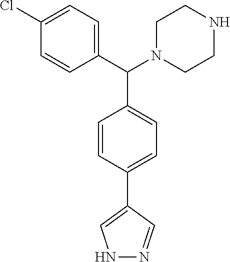 [SMILES: Clc1ccc(C(c2ccc(-c3cn[nH]c3)cc2)N2CCNCC2)cc1] |
| Methylenephenyl-substituted pyrazole based ROCK-II inhibitor | 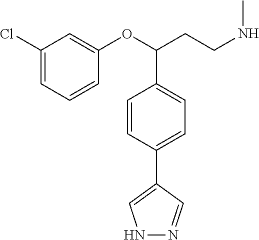 [SMILES: CNCCC(Oc1cccc(Cl)c1)c1ccc(-c2cn[nH]c2)cc1] |

Benzimidazole, benzothiazole, indole, and indazole substituted pyrazole derivatives as ROCK inhibitors

| | |
|---|---|
| Benzimidazole, benzothiazole, indole, and indazole substituted pyrazole derivatives as ROCK inhibitors | 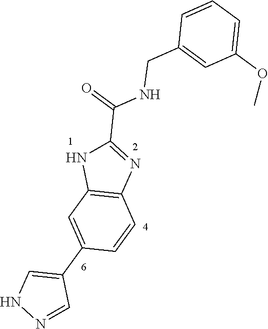 [SMILES: COc1cccc(CNC(=O)c2nc3ccc(-c4cn[nH]c4)cc3[nH]2)c1] |
| Benzimidazole, benzothiazole, indole, and indazole substituted pyrazole derivatives as ROCK inhibitors | 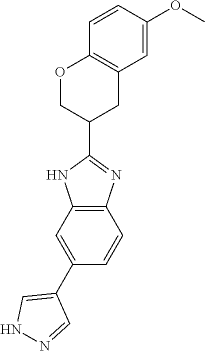 [SMILES: COc1ccc2c(c1)CC(c1nc3ccc(-c4cn[nH]c4)cc3[nH]1)CO2] |
| Benzimidazole, benzothiazole, indole, and indazole substituted pyrazole derivatives as ROCK inhibitors | 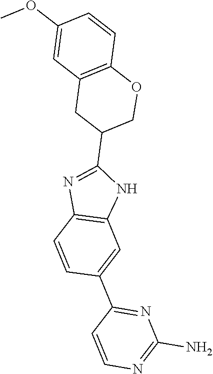 [SMILES: COc1ccc2c(c1)CC(c1nc3ccc(-c4ccnc(N)n4)cc3[nH]1)CO2] |
| Benzimidazole, benzothiazole, indole, and indazole substituted pyrazole derivatives as ROCK inhibitors | 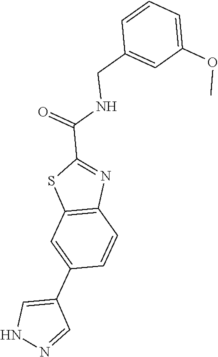 [SMILES: COc1cccc(CNC(=O)c2nc3ccc(-c4cn[nH]c4)cc3s2)c1] |
| Benzimidazole, benzothiazole, indole, and indazole substituted pyrazole derivatives as ROCK inhibitors | 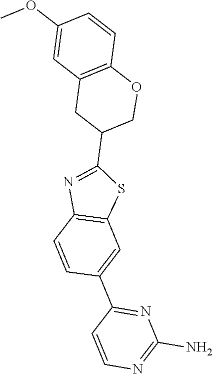 [SMILES: COc1ccc2c(c1)CC(c1nc3ccc(-c4ccnc(N)n4)cc3s1)CO2] |
| Benzimidazole, benzothiazole, indole, and indazole substituted pyrazole derivatives as ROCK inhibitors | 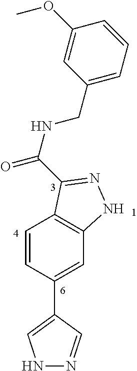 [SMILES: COc1cccc(CNC(=O)c2n[nH]c3cc(-c4cn[nH]c4)ccc23)c1] |
| Benzimidazole, benzothiazole, indole, and indazole substituted pyrazole derivatives as ROCK inhibitors | 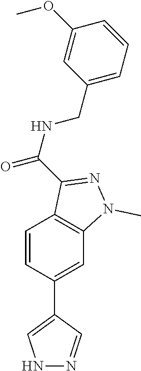 [SMILES: COc1cccc(CNC(=O)c2nn(C)c3cc(-c4cn[nH]c4)ccc23)c1] |
| Benzimidazole, benzothiazole, indole, and indazole substituted pyrazole derivatives as ROCK inhibitors | 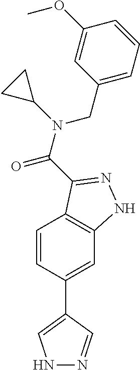 [SMILES: COc1cccc(CN(C(=O)c2n[nH]c3cc(-c4cn[nH]c4)ccc23)C2CC2)c1] |
| Benzimidazole, benzothiazole, indole, and indazole substituted pyrazole derivatives as ROCK inhibitors | 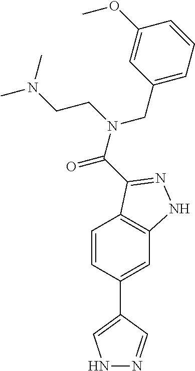 [SMILES: COc1cccc(CN(CCN(C)C)C(=O)c2n[nH]c3cc(-c4cn[nH]c4)ccc23)c1] |
| Benzimidazole, benzothiazole, indole, and indazole substituted pyrazole derivatives as ROCK inhibitors | 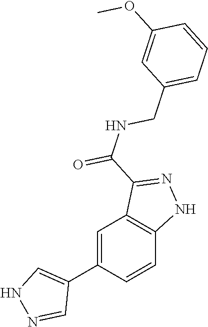 [SMILES: COc1cccc(CNC(=O)c2n[nH]c3ccc(-c4cn[nH]c4)cc23)c1] |
| Benzimidazole, benzothiazole, indole, and indazole substituted pyrazole derivatives as ROCK inhibitors | 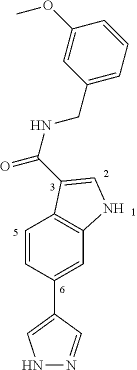 [SMILES: COc1cccc(CNC(=O)c2c[nH]c3cc(-c4cn[nH]c4)ccc23)c1] |
| Benzimidazole, benzothiazole, indole, and indazole substituted pyrazole derivatives as ROCK inhibitors | 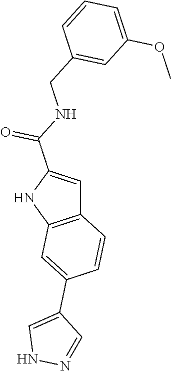 [SMILES: COc1cccc(CNC(=O)c2cc3ccc(-c4cn[nH]c4)cc3[nH]2)c1] |
| Benzimidazole, benzothiazole, indole, and indazole substituted pyrazole derivatives as ROCK inhibitors | 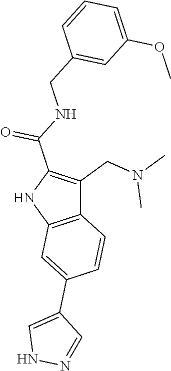 [SMILES: COc1cccc(CNC(=O)c2[nH]c3cc(-c4cn[nH]c4)ccc3c2CN(C)C)c1] |
| Benzimidazole, benzothiazole, indole, and indazole substituted pyrazole derivatives as ROCK inhibitors | 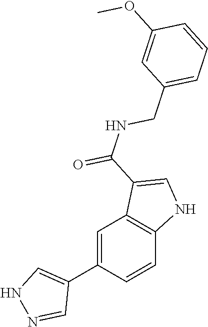 [SMILES: COc1cccc(CNC(=O)c2c[nH]c3ccc(-c4cn[nH]c4)cc23)c1] |
| Benzimidazole, benzothiazole, indole, and indazole substituted pyrazole derivatives as ROCK inhibitors | 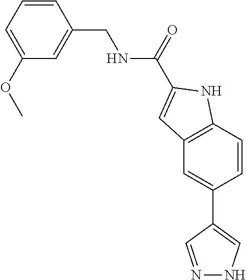 [SMILES: COc1cccc(CNC(=O)c2cc3cc(-c4cn[nH]c4)ccc3[nH]2)c1] |

Quniazolinone substituted pyrazole derivatives as ROCK inhibitors

| | |
|---|---|
| Quniazolinone substituted pyrazole derivatives as ROCK inhibitors | 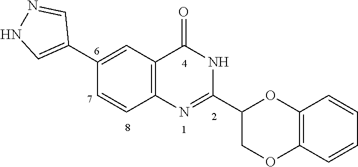 [SMILES: O=c1[nH]c(C2COc3ccccc3O2)nc2ccc(-c3cn[nH]c3)cc12] |
| Quniazolinone substituted pyrazole derivatives as ROCK inhibitors | 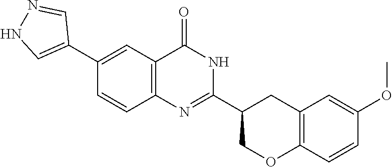 [SMILES: COc1ccc2c(c1)C[C@H](c1nc3ccc(-c4cn[nH]c4)cc3c(=O)[nH]1)CO2] |
| Quniazolinone substituted pyrazole derivatives as ROCK inhibitors | 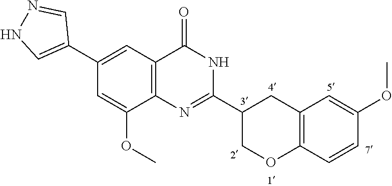 [SMILES: COc1ccc2c(c1)CC(c1nc3c(OC)cc(-c4cn[nH]c4)cc3c(=O)[nH]1)CO2] |
| Quniazolinone substituted pyrazole derivatives as ROCK inhibitors | 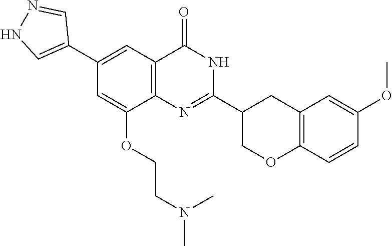 [SMILES: COc1ccc2c(c1)CC(c1nc3c(OCCN(C)C)cc(-c4cn[nH]c4)cc3c(=O)[nH]1)CO2] |
| Quniazolinone substituted pyrazole derivatives as ROCK inhibitors | 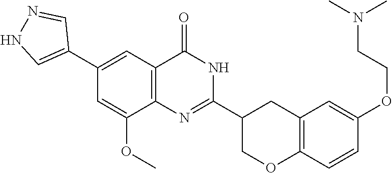 [SMILES: COc1cc(-c2cn[nH]c2)cc2c(=O)[nH]c(C3COc4ccc(OCCN(C)C)cc4C3)nc12] |
| Quniazolinone substituted pyrazole derivatives as ROCK inhibitors | 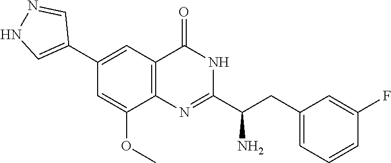 [SMILES: COc1cc(-c2cn[nH]c2)cc2c(=O)[nH]c([C@H](N)Cc3cccc(F)c3)nc12] |
| Quniazolinone substituted pyrazole derivatives as ROCK inhibitors | 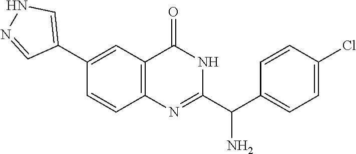 [SMILES: NC(c1ccc(Cl)cc1)c1nc2ccc(-c3cn[nH]c3)cc2c(=O)[nH]1] |
| Quniazolinone substituted pyrazole derivatives as ROCK inhibitors | 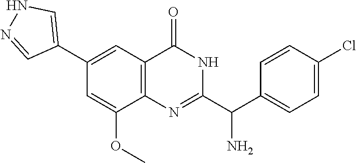 [SMILES: COc1cc(-c2cn[nH]c2)cc2c(=O)[nH]c(C(N)c3ccc(Cl)cc3)nc12] |
| Quniazolinone substituted pyrazole derivatives as ROCK inhibitors | 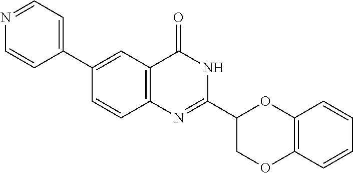 [SMILES: O=c1[nH]c(C2COc3ccccc3O2)nc2ccc(-c3ccncc3)cc12] |
| Quniazolinone substituted pyrazole derivatives as ROCK inhibitors | 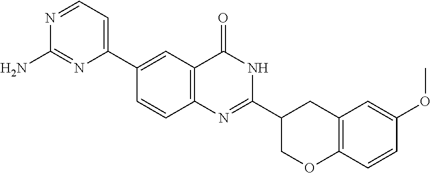 [SMILES: COc1ccc2c(c1)CC(c1nc3ccc(-c4ccnc(N)n4)cc3c(=O)[nH]1)CO2] |
| Quniazolinone substituted pyrazole derivatives as ROCK inhibitors | 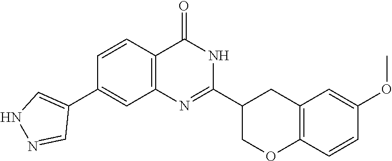 [SMILES: COc1ccc2c(c1)CC(c1nc3cc(-c4cn[nH]c4)ccc3c(=O)[nH]1)CO2] |
| Quniazolinone substituted pyrazole derivatives as ROCK inhibitors | 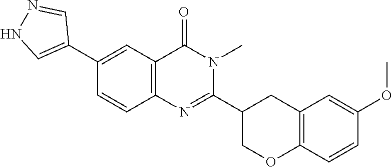 [SMILES: COc1ccc2c(c1)CC(c1nc3ccc(-c4cn[nH]c4)cc3c(=O)n1C)CO2] |
| Thiophene substituted pyrimidine based and aminofurazane based ROCK inhibitors | 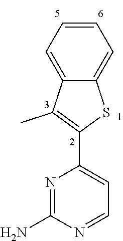 [SMILES: Cc1c(-c2ccnc(N)n2)sc2ccccc12] |
| Thiophene substituted pyrimidine based and aminofurazane based ROCK inhibitors | 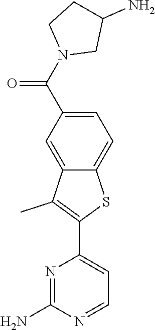 [SMILES: Cc1c(-c2ccnc(N)n2)sc2ccc(C(=O)N3CCC(N)C3)cc12] |
| Thiophene substituted pyrimidine based and aminofurazane based ROCK inhibitors | 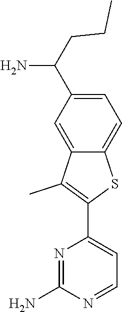 [SMILES: CCCC(N)c1ccc2sc(-c3ccnc(N)n3)c(C)c2c1] |
| Thiophene substituted pyrimidine based and aminofurazane based ROCK inhibitors | 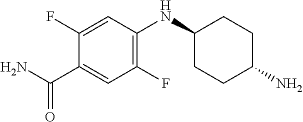 [SMILES: NC(=O)c1cc(F)c(N[C@H]2CC[C@H](N)CC2)cc1F] |
| Thiophene substituted pyrimidine based and aminofurazane based ROCK inhibitors | 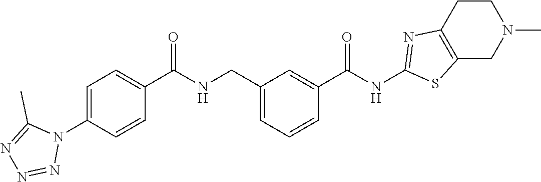 [SMILES: Cc1nnnn1-c1ccc(C(=O)NCc2cccc(C(=O)Nc3nc4c(s3)CN(C)CC4)c2)cc1] |
| Thiophene substituted pyrimidine based and aminofurazane based ROCK inhibitors | 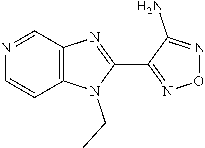 [SMILES: CCn1c(-c2nonc2N)nc2cnccc21] |
| Thiophene substituted pyrimidine based and aminofurazane based ROCK inhibitors | 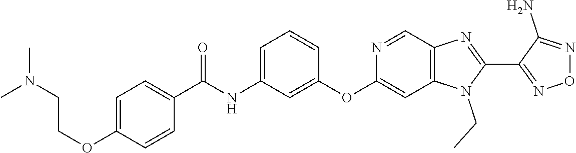 [SMILES: CCn1c(-c2nonc2N)nc2cnc(Oc3cccc(NC(=O)c4ccc(OCCN(C)C)cc4)c3)cc21] |
| Thiophene substituted pyrimidine based and aminofurazane based ROCK inhibitors | 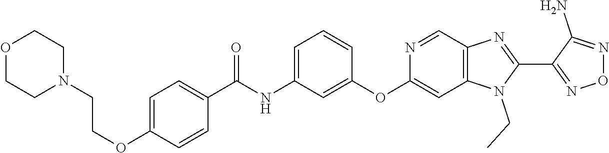 [SMILES: CCn1c(-c2nonc2N)nc2cnc(Oc3cccc(NC(=O)c4ccc(OCCN5CCOCC5)cc4)c3)cc21] |

6- and 7-substituted isoquinolines and isoquinolinones based ROCK inhibitors.

| | |
|---|---|
| Aerie's ROCK Inhibitors | 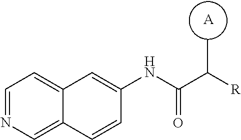 |
| | Ring A is an aromatic moiety |
| | R is an alkyl chain containing an amino moiety |
| Sanofi-Aventis ROCK Inhibitors | 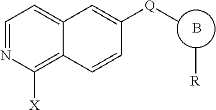 |
| | 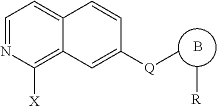 |
| | Ring B is a 4- to 7-membered staurated ring |
| | R is an alkyl chain optionally substituted by an aromatic moeity |
| | X = H, OH |
| | Q = O, NH, S |

Boron-derivatives as ROCK Inhibitors

| | |
|---|---|
| Boron-derivatives as ROCK Inhibitors | 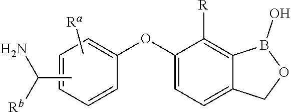 |
| Boron-derivatives as ROCK Inhibitor | 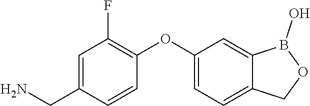 [SMILES: NCc1ccc(Oc2ccc3c(c2)B(O)OC3)c(F)c1] |
| Boron-derivatives as ROCK Inhibitor | 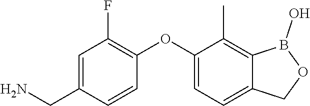 [SMILES: Cc1c(Oc2ccc(CN)cc2F)ccc2c1B(O)OC2] |
| Boron-derivatives as ROCK Inhibitor | 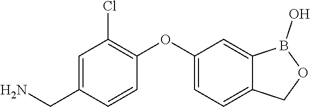 [SMILES: NCc1ccc(Oc2ccc3c(c2)B(O)OC3)c(Cl)c1] |
| Boron-derivatives as ROCK Inhibitor | 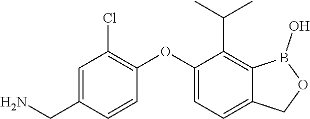 [SMILES: CC(C)c1c(Oc2ccc(CN)cc2Cl)ccc2c1B(O)OC2] |
| Boron-derivatives as ROCK Inhibitor | 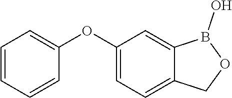 [SMILES: OB1OCc2ccc(Oc3ccccc3)cc21] |

Amakem’s Soft ROCK Inhibitors

| | |
|---|---|
| Amakem’s Soft ROCK Inhibitor | 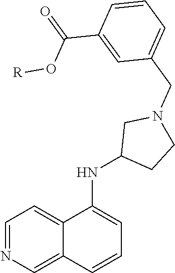 |
| Amakem’s Soft ROCK Inhibitor | 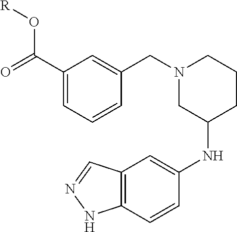 |
| Amakem’s Soft ROCK Inhibitor | 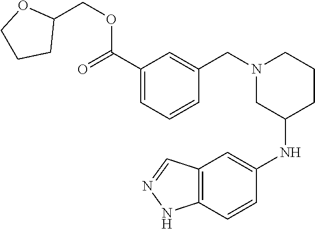 [SMILES: O=C(OCC1CCCO1)c1cccc(CN2CCCC(Nc3ccc4[nH]ncc4c3)C2)c1] |
| Amakem’s Soft ROCK Inhibitor | 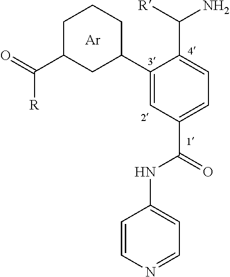 |
| Amakem™’s Soft ROCK Inhibitor | 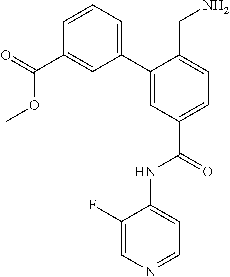 [SMILES: COC(=O)c1cccc(-c2cc(C(=O)Nc3ccncc3F)ccc2CN)c1] |
| Amakem’s Soft ROCK Inhibitor | 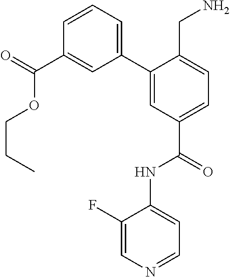 [SMILES: CCCOC(=O)c1cccc(-c2cc(C(=O)Nc3ccncc3F)ccc2CN)c1] |
| Amakem’s Soft ROCK Inhibitor | 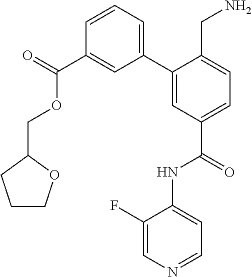 [SMILES: NCc1ccc(C(=O)Nc2ccncc2F)cc1-c1cccc(C(=O)OCC2CCCO2)c1] |
| Amakem’s Soft ROCK Inhibitor | 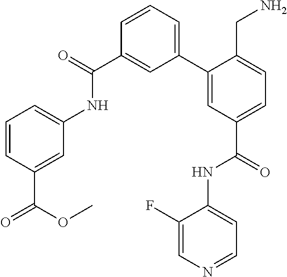 [SMILES: COC(=O)c1cccc(NC(=O)c2cccc(-c3cc(C(=O)Nc4ccncc4F)ccc3CN)c2)c1] |
| Amakem’s Soft ROCK Inhibitor | 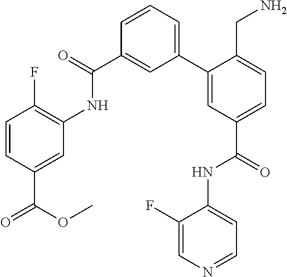 [SMILES: COC(=O)c1ccc(F)c(NC(=O)c2cccc(-c3cc(C(=O)Nc4ccncc4F)ccc3CN)c2)c1] |
| Amakem’s Soft ROCK Inhibitor | 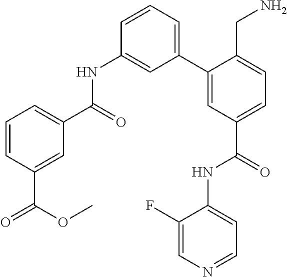 [SMILES: COC(=O)c1cccc(C(=O)Nc2cccc(-c3cc(C(=O)Nc4ccncc4F)ccc3CN)c2)c1] |
| Amakem’s Soft ROCK Inhibitor | 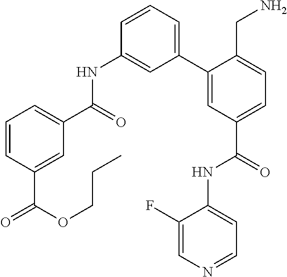 [SMILES: CCCOC(=O)c1cccc(C(=O)Nc2cccc(-c3cc(C(=O)Nc4ccncc4F)ccc3CN)c2)c1] |
| Amakem’s Soft ROCK Inhibitor | 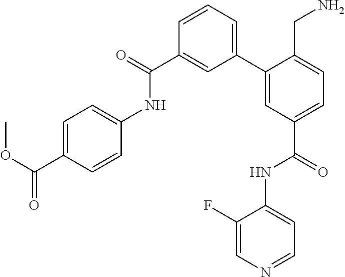 [SMILES: COC(=O)c1ccc(NC(=O)c2cccc(-c3cc(C(=O)Nc4ccncc4F)ccc3CN)c2)cc1] |
| Amakem’s Soft ROCK Inhibitor | 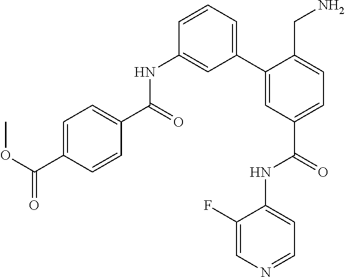 [SMILES: COC(=O)c1ccc(C(=O)Nc2cccc(-c3cc(C(=O)Nc4ccncc4F)ccc3CN)c2)cc1] |
| Amakem’s Soft ROCK Inhibitor | 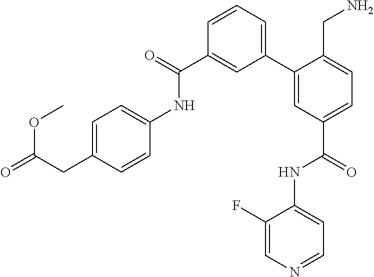 [SMILES: COC(=O)Cc1ccc(NC(=O)c2cccc(-c3cc(C(=O)Nc4ccncc4F)ccc3CN)c2)cc1] |
| Amakem’s Soft ROCK Inhibitor | 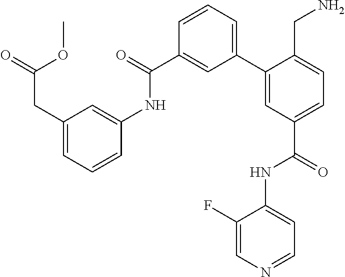 [SMILES: COC(=O)Cc1cccc(NC(=O)c2cccc(-c3cc(C(=O)Nc4ccncc4F)ccc3CN)c2)c1] |

Isoform selective ROCK-II inhibitors from Kadmon

| | |
|---|---|
| Isoform selective ROCK-II inhibitor from Kadmon | 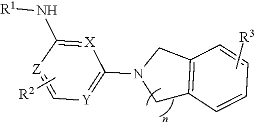 |
| Isoform selective ROCK-II inhibitor from Kadmon | 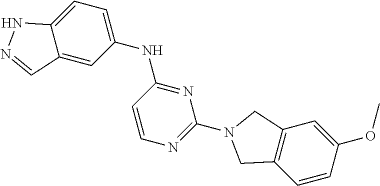 [SMILES: COc1ccc2c(c1)CN(c1nccc(Nc3ccc4[nH]ncc4c3)n1)C2] |
| Isoform selective ROCK-II inhibitor from Kadmon | 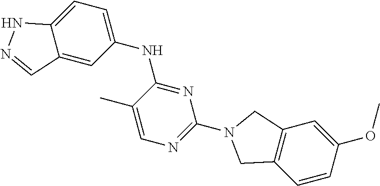 [SMILES: COc1ccc2c(c1)CN(c1ncc(C)c(Nc3ccc4[nH]ncc4c3)n1)C2] |
| Isoform selective ROCK-II inhibitor from Kadmon | 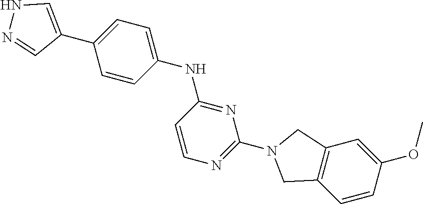 [SMILES: COc1ccc2c(c1)CN(c1nccc(Nc3ccc(-c4cn[nH]c4)cc3)n1)C2] |
| Isoform selective ROCK-II inhibitor from Kadmon | 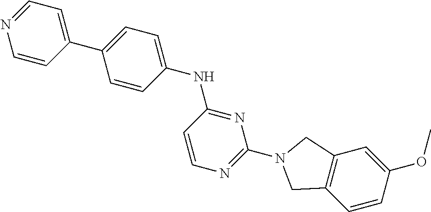 [SMILES: COc1ccc2c(c1)CN(c1nccc(Nc3ccc(-c4ccncc4)cc3)n1)C2] |

ROCK inhibitors from Bristol Myers Squib

| | |
|---|---|
| ROCK inhibitor from Bristol Myers Squib | 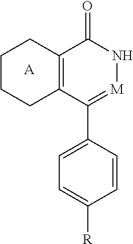 |
| ROCK inhibitor from Bristol Myers Squib | 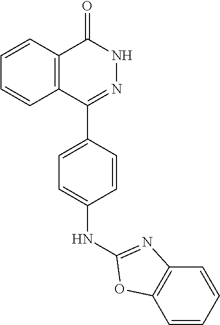 [SMILES: O=c1[nH]nc(-c2ccc(Nc3nc4ccccc4o3)cc2)c2ccccc12] |
| ROCK inhibitor from Bristol Myers Squib | 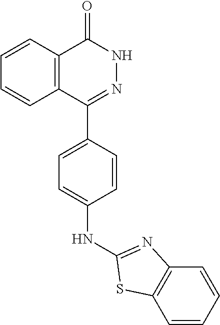 [SMILES: O=c1[nH]nc(-c2ccc(Nc3nc4ccccc4s3)cc2)c2ccccc12] |
| ROCK inhibitor from Bristol Myers Squib | 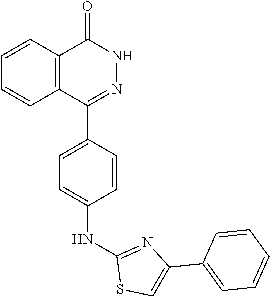 [SMILES: O=c1[nH]nc(-c2ccc(Nc3nc(-c4ccccc4)cs3)cc2)c2ccccc12] |
| ROCK inhibitor from Bristol Myers Squib | 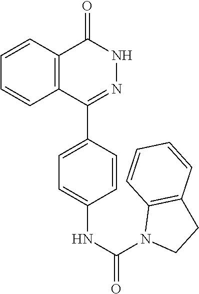 [SMILES: O=C(Nc1ccc(-c2n[nH]c(=O)c3ccccc23)cc1)N1CCc2ccccc21] |

Advanced ROCK inhibitors

| | |
|---|---|
| K-115 | 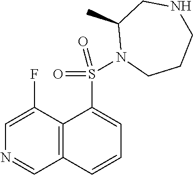 [SMILES: C[C@H]1CNCCCN1S(=O)(=O)c1cccc2cncc(F)c12] |
| AR-12141 | 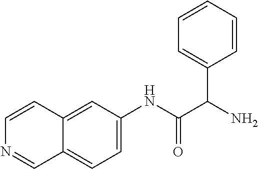 [SMILES: NC(C(=O)Nc1ccc2cnccc2c1)c1ccccc1] |
| AR-12432 | 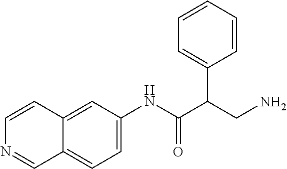 [SMILES: NCC(C(=O)Nc1ccc2cnccc2c1)c1ccccc1] |
| INS117548 | 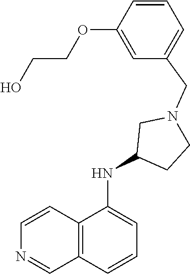 |
| AT13148 | 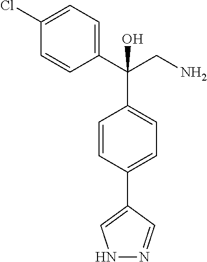 [SMILES: NC[C@@](O)(c1ccc(Cl)cc1)c1ccc(-c2cn[nH]c2)cc1] |
| RKI1447 | 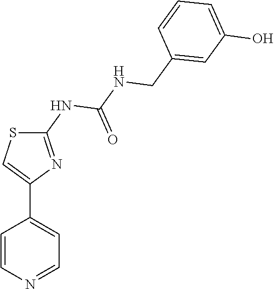 |
| SAR407899 | 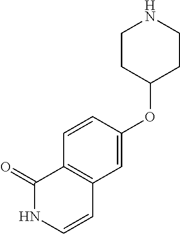 [SMILES: O=c1[nH]ccc2cc(OC3CCNCC3)ccc12] |

In one embodiment, the ROCK inhibitor Y-27632 has the structure of Formula

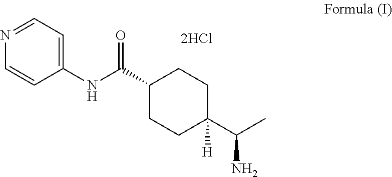 [SMILES: C[C@@H](N)[C@H]1CC[C@H](C(=O)Nc2ccncc2)CC1]

(I):

In some embodiments, a ROCK inhibitor is contacted with the stem cell in a differentiation program beginning before differentiation is initiated, and then maintained in contact with the differentiating cells or culture throughout the program. In vitro differentiation of stem cells to cardiomyocytes is discussed further herein below, but generally proceeds in the following order: ESC or iPSC>cardiogenic mesoderm>cardiac progenitor cells>cardiomyocytes (see e.g., US20170240861, the contents of which are incorporated herein by reference in its entirety; it is specifically contemplated that inclusion of a ROCK inhibitor during differentiation as described herein can benefit the viability of cardiomyocytes differentiated with protocols that differ from that described in the '861 document). As described herein, “day zero” of a differentiation program is the day the first differentiation-promoting agent(s) is (are) contacted with the stem cells. Thus, in some embodiments, a ROCK inhibitor can be contacted with the stem cells beginning before the first differentiation-promoting agent(s) is (are) added at day zero. One or more ROCK inhibitors can be in contact with the differentiating cell for at least zero days (first day of differentiation), at least one day, at least two days, at least three days, at least four days, at least five days, at least 6 days, at least 7 days, at least 8 days, at least 9 days, at least 10 days, at least 11 days, at least 12 days, at least 13 days, at least 14 days or more. In some embodiments, a stem cell is contacted with at least one ROCK inhibitor throughout the course of differentiation of the stem cell to a cardiomyocyte. In some embodiments, the contact with at least one ROCK inhibitor is initiated when a Wnt modulator is added during the differentiation protocol, and maintained throughout subsequent differentiation. In some embodiments, the contact with at least one ROCK inhibitor is initiated when a Wnt modulator is added during the differentiation protocol, and maintained for at least 50% of the time, at least 60% of the time, at least 70% of the time, at least 80% of the time, at least 90% of the time, at least 95% of the time or more until differentiation to cardiomyocytes is complete. Fully or completely differentiated cardiomyocytes express cardiac troponin T (cTnT). Fully differentiated cardiomyocytes are also generally contractile.

Where differentiation generally proceeds in the order: ESC or iPSC>cardiogenic mesoderm>cardiac progenitor cells>cardiomyocytes, a ROCK inhibitor can be present, for example, in the step of differentiation from ESC or iPSC to cardiogenic mesoderm. In some embodiments, the ROCK inhibitor is present at least 80%, at least 90% at least 95%, at least 96%, at least 97%, at least 98%, at least 99% or more, including 100% of the time during which ESCs or iPSCs differentiate to cardiogenic mesoderm. Markers for cardiogenic mesoderm are known to those of ordinary skill in the art, and include, for example, mesoderm posterior 1 (Mesp1). A ROCK inhibitor can also be present during the transition from cardiogenic mesoderm to cardiac progenitor cell. In some embodiments, the ROCK inhibitor is present at least 80%, at least 90% at least 95%, at least 96%, at least 97%, at least 98%, at least 99% or more, including 100% of the time during which cardiogenic mesoderm cells differentiate to cardiac progenitor cells. Markers for cardiac progenitor cells are known to those of ordinary skill in the art, and include, for example, G protein-coupled receptor 4 (GPR4)—see, e.g., Torán et al., Scientific Reports 9:467 (2019). A ROCK inhibitor can also be present during the transition from cardiac progenitor cell to cardiomyocyte. In some embodiments, the ROCK inhibitor is present at least 80%, at least 90% at least 95%, at least 96%, at least 97%, at least 98%, at least 99% or more, including 100% of the time during which cardiac progenitor cells differentiate to cardiomyocytes. Markers for cardiomyocytes are known to those of ordinary skill in the art, and include, for example, cardiac troponin T (cTnT).

In some embodiments, the ROCK inhibitor is present in the media at a concentration of at least between 0.1 μM-100 mM, between 0.1 μM-95 mM, between 0.1 μM-90 mM, between 0.1 μM-85 mM, between 0.1 μM-80 mM, between 0.1 μM-75 mM, between 0.1 μM-70 mM, between 0.1 μM-65 mM, between 0.1 μM-60 mM, between 0.1 μM-55 mM, between 0.1 μM-50 mM, between 0.1 μM-45 mM, between 0.1 μM-40 mM, between 0.1 μM-35 mM, between 0.1 μM-30 mM, between 0.1 μM-25 mM, between 0.1 μM-20 mM, between 0.1 μM-15 mM, between 0.1 μM-10 mM, between 0.1 μM-5 mM, between 0.1 μM-1 mM, between 0.1 μM-0.9 mM, between 0.1 μM-0.8 mM, between 0.1 μM-0.7 mM, between 0.1 μM-0.6 μM, between 0.1 μM-0.5 mM, between 0.1 μM-0.4 mM, between 0.1 μM-0.3 mM, between 0.1 μM-0.2 mM, between 0.5 μM-100 mM, between 1 μM-100 mM, between 5 μM-100 mM, between 10 μM-100 mM, between 15 μM-100 mM, between 20 μM-100 mM, between 25 μM-100 mM, between 30 μM-100 mM, between 35 μM-100 mM, between 40 μM-100 mM, between 45 μM-100 mM, between 50 μM-100 mM, between 55 μM-100 mM, between 60 μM-100 mM, between 65 μM-100 mM, between 70 μM-100 mM, between 75 μM-100 mM, between 80 μM-100 mM, between 85 μM-100 mM, between 90 μM-100 mM, between 95 μM-100 mM, between 0.1 mM-100 mM, between 0.1 mM-95 mM, between 0.1 mM-90 mM, between 0.1 mM-85 mM, between 0.1 mM-80 mM, between 0.1 mM-75 mM, between 0.1 mM-70 mM, between 0.1 mM-65 mM, between 0.1 mM-60 mM, between 0.1 mM-55 mM, between 0.1 mM-50 mM, between 0.1 mM-45 mM, between 0.1 mM-40 mM, between 0.1 mM-35 mM, between 0.1 mM-30 mM, between 0.1 mM-25 mM, between 0.1 mM-20 mM, between 0.1 mM-15 mM, between 0.1 mM-10 mM, between 0.1 mM-5 mM, between 0.1 mM-1 mM, between 0.1 mM-0.9 mM, between 0.1 mM-0.8 mM, between 0.1 mM-0.7 mM, between 0.1 mM-0.6 mM, between 0.1 mM-0.5 mM, between 0.1 mM-0.4 mM, between 0.1 mM-0.3 mM, between 0.1 mM-0.2 mM, between 0.5 mM-100 mM, between 1 mM-100 mM, between 5 mM-100 mM, between 10 mM-100 mM, between 15 mM-100 mM, between 20 mM-100 mM, between 25 mM-100 mM, between 30 mM-100 mM, between 35 mM-100 mM, between 40 mM-100 mM, between 45 mM-100 mM, between 50 mM-100 mM, between 55 mM-100 mM, between 60 mM-100 mM, between 65 mM-100 mM, between 70 mM-100 mM, between 75 mM-100 mM, between 80 mM-100 mM, between 85 mM-100 mM, between 90 mM-100 mM, between 95 mM-100 mM between 0.1 μM-100 μM, between 0.1 μM-95 μM, between 0.1 μM-90 μM, between 0.1 μM-85 μM, between 0.1 μM-80 μM, between 0.1 μM-75 μM, between 0.1 μM-70 μM, between 0.1 μM-65 μM, between 0.1 μM-60 μM, between 0.1 μM-55 μM, between 0.1 μM-50 μM, between 0.1 μM-45 μM, between 0.1 μM-40 μM, between 0.1 μM-35 μM, between 0.1 μM-30 μM, between 0.1 μM-25 μM, between 0.1 μM-20 μM, between 0.1 μM-15 μM, between 0.1 μM-10 μM, between 0.1 μM-5 μM, between 0.1 μM-1 μM, between 0.1 μM-0.9 μM, between 0.1 μM-0.8 μM, between 0.1 μM-0.7 μM, between 0.1 μM-0.6 μM, between 0.1 μM-0.5 μM, between 0.1 μM-0.4 μM, between 0.1 μM-0.3 μM, between 0.1 μM-0.2 μM, between 0.5 μM-100 μM, between 1 μM-100 μM, between 5 μM-100 μM, between 10 μM-100 μM, between 15 μM-100 μM, between 20 μM-100 μM, between 25 μM-100 μM, between 30 μM-100 μM, between 35 μM-100 μM, between 40 μM-100 μM, between 45 μM-100 μM, between 50 μM-100 μM, between 55 μM-100 μM, between 60 μM-100 μM, between 65 μM-100 μM, between 70 μM-100 μM, between 75 μM-100 μM, between 80 μM-100 μM, between 85 μM-100 μM, between 90 μM-100 μM, between 95 μM-100 μM between 0.5 μM-40 μM, between 1 μM-30 μM, between 5 μM-20 μM, between 1 μM-10 μM, between 5 μM-10 μM, between 5 μM-40 μM, between 5 μM-30 μM, between 5 μM-25 μM, between 5 μM-20 μM, between 1 μM-10 μM, between 1 μM-5 μM.

ROCK inhibitor Y-27632 is available commercially. Exemplary sources include, but are not limited to, Y27632 2HCL (Cat. No. S1049, Selleck Chem, Houston, TX); Y-27632 (Cat. No. B1293, APExBio, Houston, TX); ROCK inhibitor (Y-27632) (Cat. No. SCM075, Sigma Aldrich, St. Louis, MO). Additional examples can be found at J. Med. Chem. 2016, 59, 2269-2300, which is incorporated herein in its entirety.

ROCK inhibitors can induce aggregation of stem cells. If cell concentration and/or ROCK inhibitor concentration is high, ROCK inhibitor can induce the formation of large clumps of cells, and cells in the interior of the clumps may experience conditions sufficiently different from those on or near the outside that cell viability and/or differentiation are affected. Cells in the interior of large clumps can lose viability, which affects the overall yield of differentiated cells. While not wishing to be bound by theory, cells in the interior of such clumps may lose or have reduced contact with the ROCK inhibitor and thus not benefit from the protective effect of the ROCK inhibitor demonstrated herein. On the other hand, if the concentration of cells is low, the cells may fail to aggregate or to adhere to substrate and lose viability. The concentration of the cells and/or the concentration of the ROCK inhibitor can be titrated to mitigate these issues. For example, excessive aggregation of cells can be remedied by reducing the dose of ROCK inhibitor (e.g., less than 20 μM inhibitor for 5-5000 cells), and insufficient aggregation or adherence due to reduced cell numbers (e.g., during single cell cloning) can be remedied with a higher dose of ROCK inhibitor (e.g., more than 0.1 μM of inhibitor for 5-5000 cells).

### Wnt Signaling (Activators and Inhibitors)

The hallmark of canonical Wnt signaling activation is elevated levels of the protein β-catenin. β-catenin, which is a transcription factor, is constitutively produced and is present in the cytoplasm as pools of monomeric protein. (See e.g., Papkoff, J. et al., Mol. Cell Biol. 1996; 16:2128-2134). The primary mechanism for controlling cytoplasmic levels of β-catenin is through direct physical degradation upon recruitment into a large multi-protein complex (“degradation complex”). After formation, the complex is stabilized by the GSK3β-mediated phosphorylation of the protein components Axin and APC, as well as PP2A. GSK3β-then phosphorylates β-catenin, thereby allowing it to be recognized by β-transducin repeat containing protein (β-TrCP), and targeting it for ubiquitination and proteosomic degradation. (See e.g., Aberle et al., EMBO J. 1997; 16:3797-804; Latres et al., Oncogene 1999; 18:849-54; Liu et al., Proc. Natl. Acad. Sci. USA 1999; 96:6273-8). An alternative degradation pathway has been shown involving ubiquitination induced by complexation with Siah-1 and the C-terminus of APC. (Matsuzawa et al., Mol Cell 2001; 7:915-926; Liu et al., Mol. Cell 2001; 7:927-936). In addition to its role as a transcription factor, β-catenin further is involved in cellular adhesion. [Nelson et al., Science 2004; 303:1483-1487; Ilyas et al., J. Pathol. 1997; 182:128-137.

β-catenin can be found at the cell surface sites of intercellular contact known as adherens junctions, where it is complexed with E-cadherin. Thus, the breakdown of the E-cadherin-catenin complex can increase cytoplasmic levels of free β-catenin, thereby stimulating transcriptional activity. Activation of the cell surface receptors cRON, epidermal growth factor receptor (EGFR) and c-ErbB2, by liberating β-catenin, can also stimulate canonical Wnt signaling. Other signaling pathways can either activate or facilitate the effects of Wnt signaling. For example, signaling through insulin-like growth factor (IGF) can activate Wnt signaling by “soaking up” available GSK3β-thereby preventing formation of the “degradation complex.”

As used herein, the term “Wnt agonist” refers to any agent that activates the Wnt/β-catenin pathway, or inhibits the activity and/or expression of inhibitors of Wnt/β-catenin signaling, for example antagonists or inhibitors of GSK-3β activity. A Wnt activating agent as used herein can enhance signaling through the Wnt/β-catenin pathway at any point along the pathway, for example, but not limited to increasing the expression and/or activity of Wnt, or β-catenin or Wnt dependent genes and/or proteins, and decreasing the expression and/or activity of endogenous inhibitors of Wnt and/or β-catenin or decreasing the expression and/or activity of endogenous inhibitors of components of the Wnt/β-catenin pathway, for example decreasing the expression of GSK-3B.

Some non-limiting examples of Wnt pathway agonists include GSK-3β inhibitors (e.g., CHIR99201), TCS2002, TWS119, SB-216763, BIO and lithium chloride. In some embodiments, the exemplary Wnt activators comprise: CHIR99201, TCS2002, TWS119, SB-216763, BIO, and Lithium chloride.

In one embodiment, the CHIR99021 GSK-3B inhibitor has the structure of Formula (II):

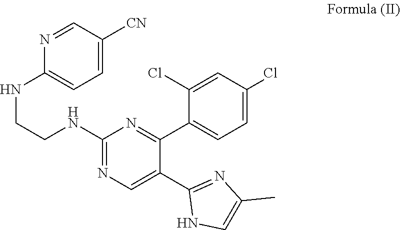 [SMILES: Cc1c[nH]c(-c2cnc(NCCNc3ccc(C#N)cn3)nc2-c2ccc(Cl)cc2Cl)n1]

In another embodiment, the GSK-3B inhibitor BIO has the structure of Formula (III):

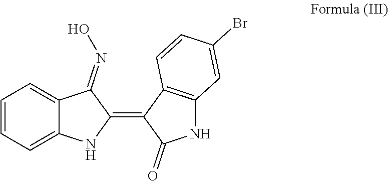 [SMILES: O=C1Nc2cc(Br)ccc2/C1=C1/Nc2ccccc2/C1=N\O]

In another embodiment, the GSK-3B inhibitor SB-216763 has the structure of Formula (IV):

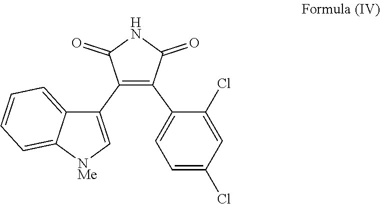 [SMILES: Cn1cc(C2=C(c3ccc(Cl)cc3Cl)C(=O)NC2=O)c2ccccc21]

Without wishing to be bound by theory, Wnt proteins and their cognate receptors signal through at least two distinct intracellular pathways. The “canonical” Wnt signaling pathway, (referred to herein as the Wnt/β-catenin pathway) involves Wnt signaling via β-catenin to activate transcription through TCF-related proteins (van de Wetering et al. (2002) Cell 109 Suppl: S13-9; Moon et al. (2002) Science 296 (5573): 1644-6). A non-canonical alternative pathway exists, in which Wnt activates protein kinase C (PKC), calcium/calmodulin-dependent kinase II (CaMKII), JNK and Rho-GTPases (Veeman et al. (2003) Dev Cell 5 (3): 367-77), and is often involved in the control of cell polarity.

As used herein, the term “Wnt antagonist” or “Wnt inhibitor” refers to any agent that inhibits the Wnt/β-catenin pathway, or enhances the activity and/or expression of inhibitors of Wnt/β-catenin signaling, for example activators or enhancers of GSK-3B activity. A Wnt inhibitory agent as used herein can suppress the Wnt/β-catenin pathway at any point along the pathway, for example, but not limited to decreasing the expression and/or activity of Wnt, or β-catenin or Wnt dependent genes and/or proteins, and increasing the expression and/or activity of endogenous inhibitors of Wnt and/or β-catenin or increasing the expression and/or activity of endogenous inhibitors of components of the Wnt/β-catenin pathway, for example increasing the expression of GSK-3B or inhibiting the tankyrase enzyme.

Some non-limiting examples of Wnt antagonists include Wnt pathway inhibitor WIKI4, XAV939 (tankyrase inhibitor), E7449, AZ6102, and JW55.

In another embodiment, the WIKI4 tankyrase inhibitor has the structure of Formula (V):

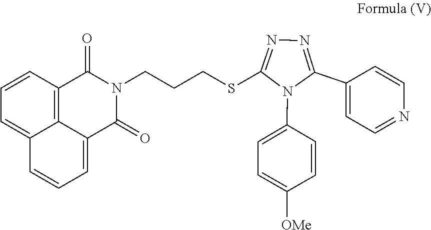 [SMILES: COc1ccc(-n2c(SCCCN3C(=O)c4cccc5cccc(c45)C3=O)nnc2-c2ccncc2)cc1]

In another embodiment, the XAV-939 tankyrase inhibitor has the structure of Formula (VI):

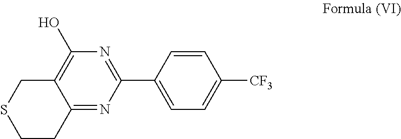 [SMILES: Oc1nc(-c2ccc(C(F)(F)F)cc2)nc2c1CSCC2]

In another embodiment, the IWR tankyrase inhibitor has the structure of Formula (VII):

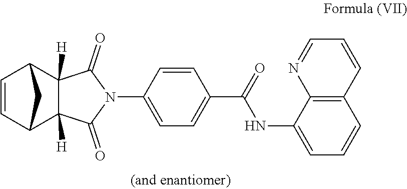 [SMILES: O=C(Nc1cccc2cccnc12)c1ccc(N2C(=O)[C@@H]3[C@H](C2=O)[C@@H]2C=C[C@H]3C2)cc1]

In another embodiment, the JW55 tankyrase inhibitor has the structure of Formula (VIII):

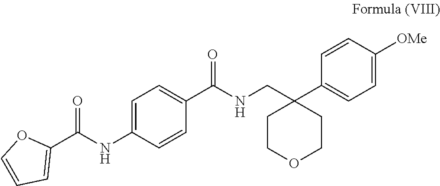 [SMILES: COc1ccc(C2(CNC(=O)c3ccc(NC(=O)c4ccco4)cc3)CCOCC2)cc1]

In some embodiments, the dosage range useful for a Wnt antagonist (e.g. WIKI4) is between 0.5 and 5 μM, between 0.5 and 4 μM, between 0.5 and 3 μM, between 0.5 and 2 μM, between 0.5 and 1 μM, between 4 and 5 μM, between 3 and 5 μM, between 2 and 5 μM, between 1 and μM, between 0.5 and 2 μM, between 0.75 and 2 μM, between 0.9 μM and 2 μM, or any range therebetween.

In some embodiments the dose of a Wnt antagonist is e.g., at least 20 ng/ml, at least 30 ng/mL, at least 40 ng/mL, at least 50 ng/ml, at least 60 ng/ml, at least 70 ng/ml, at least 80 ng/ml, at least 90 ng/ml, at least 100 ng/mL, at least 110 ng/mL, at least 120 ng/ml, at least 130 ng/ml, at least 140 ng/mL, at least 150 ng/mL, at least 160 ng/ml, at least 170 ng/ml, at least 180 ng/ml, at least 190 ng/ml, at least 200 ng/ml, or more.

It is demonstrated herein that in vitro cardiomyocyte differentiation cell losses associated with the use of Wnt modulators, including the Wnt activator CHIR99021 and the Wnt inhibitor WIKI4, can be mitigated by inclusion of one or more ROCK inhibitors during differentiation. It is specifically contemplated that any in vitro cardiomyocyte differentiation program using one or more Wnt modulators described herein or known in the art can benefit from the use of one or more ROCK inhibitors, including those described herein or known in the art. In vitro differentiation of stem cells to cardiomyocytes is discussed further herein below.

### Immunosuppressants

As used herein, an “immunosuppressant” is a drug that inhibits or prevents activity of the immune system. There are different groups of immunosuppressants, including, but not limited to glucocorticoids, cytostatics, certain antibodies, drugs acting on immunophilins, and other drugs. Immunosuppressants can help to limit or avoid acute transplant rejection of an organ (e.g., heart), tissue or cells (e.g., cardiomyocytes). In some embodiments, an immunosuppressant is selected from glucocorticoids and drugs acting on immunophilins. In some embodiments, an immunosuppresant includes Cyclosporine A (anti-inflammatory, calcineurin inhibitor), corticosteroid agents (prednisone, methylprednisolone etc.), mycophenolate mofetil (MMF), and/or Tacrolimus.

### Apoptosis Inhibitors

As used herein, an “apoptosis inhibitor” is a molecule, drug, or protein that blocks or inhibits programmed cell death, or apoptosis. Apoptosis inhibitors include, for example, caspase inhibitors. Caspases are a family of cysteine proteases that play an essential role in apoptosis. Caspase inhibitors can include selective inhibitors, which inhibit one or only a limited subset of specific caspases, or broad-spectrum or so-called “pan-caspase” inhibitors, which, as the name implies, inhibit the caspase enzymes more broadly. Selective caspase inhibitors include, as non-limiting examples, belnacasan (VX-765; caspase 1 inhibitor), Z-DEVD-FMK (caspase 3 inhibitor) and Z-IETD-FMK (caspase 8 inhibitor), among others. Pan-caspase inhibitors include, but are not limited to Q-VD-Oph, Z-VAD-FMK, emricasan, and Z-VAD (OH)-FMK, among others. Other apoptosis inhibitors target different components of the apoptotic pathway and include, for example, cytochrome C inhibitors (e.g., minocycline, methazolimide, gamma-tocotrienol, 3-hydroxypropyl-triphenylphosphonium-conjugated imidazole-substituted oleic acid (TPP-IOA), 3-hydroxypropyl-triphenylphosphonium-conjugated imidazole-substituted stearic acid (TPP-ISA), and TPP-6-ISA, among others), BH3-interacting domain death agonist (BID) inhibitors (e.g., BI-6C9, TC9-305, BI-11A7, 3-O-tolylthiazolidine-2,4-dione, among others), Fas inhibitors (e.g., KR-33493, RKTS-33, dichlorovinyl dimethylphosphate (DDVP), geldanamycin, vitamin D3, cilazapril, M50054, vanillic acid, NCX-1000, among others), Tumor Necrosis Factor (TNF) inhibitors (e.g., infliximab, etanercept, adalimumab, golimumab, and small molecule compounds noted in section 1.2.1 of Abdelhafez et al., chapter “Apoptotic Inhibitors as Therapeutic Targets for Cell Survival” in Cytotoxicity-Definition, Identification, and Cytotoxic Compounds, ISBN 978-1-78984-755-0 (2019)) among others, p53-upregulated modulator of apoptosis (PUMA) inhibitors (e.g., CLZ-8, among others), and Bax inhibitors (e.g., PD98059, vitamin E, tanshinone, among others), among others.

### K-ATP Channel Drug

As used herein, a “potassium channel opener” or “K-ATP channel drug” is a type of drug which facilitates ion transmission through potassium channels. There are 4 classes and 14 subclasses of K-ATP channel drugs. Examples of K-ATP channel drugs include, but are not limited to Pinacidil, Diazoxide, Nicorandil, and BNS180448.

### In Vitro Differentiation of Cardiomyocytes

The methods and compositions described herein can use grafts comprising in vitro-differentiated cardiomyocytes. Methods for the differentiation of cardiomyocytes from ESCs or iPSCs are known in the art. See, e.g., LaFlamme et al., Nature Biotech 25:1015-1024 (2007), (which describes the differentiation of cardiomyocytes from ESCs), and Batalov & Feinberg, Biomarker Insights 10:71-76 (2015), (which reviews various approaches for differentiation of cardiomyocytes from ESCs and iPSCs). Exemplary methods for generating cardiomyocytes or cardiac progenitor cells from pluripotent stem cells (e.g., embryonic stem cells or induced pluripotent stem cells) are also described in e.g., US2020-0085880, the contents of which are incorporated herein by reference in their entirety.

Various other methods of differentiating stem cells to cardiomyocytes are known to those of ordinary skill in the art; see, e.g., Lyra-Leite et al., STAR Protoc. 3:101560 (2022), “A review of protocols for human iPSC culture, cardiac differentiation, subtype specification, maturation, and direct reprogramming,” which is incorporated herein by reference, for a review —see entire reference, and references cited therein; see particularly Tables 1 and 2 therein.

These approaches use various factors and conditions to activate and guide differentiation programs leading to their respective cell types. Pathways and certain of the factors involved in them are discussed in the following.

In certain embodiments, the step-wise differentiation of ESCs or iPSCs to cardiomyocytes proceeds in the following order: ESC or iPSC>cardiogenic mesoderm>cardiac progenitor cells>cardiomyocytes (see e.g., US20170240861, the contents of which are incorporated herein by reference in its entirety).

As will be appreciated by those of skill in the art, in vitro-differentiation of cardiomyocytes produces an end-result of a cell having the phenotypic and morphological features of a cardiomyocyte but the differentiation steps of in vitro-differentiation need not be the same as the differentiation that occurs naturally in the embryo. That is, during differentiation to a cardiomyocyte, it is specifically contemplated herein that the step-wise differentiation approach utilized to produce such cells need not proceed through every progenitor cell type that has been identified during embryogenesis and can essentially “skip” over certain stages of development that occur during embryogenesis.

The technology may be as described in any one of the following numbered paragraphs:

Paragraph 1. An in vitro differentiation method for preparing a cardiomyocyte from a stem cell, the method comprising contacting the stem cell with an inhibitor of Rho-associated, coiled-coil containing protein kinase (ROCK) throughout differentiation from stem cell to cardiomyocyte.

Paragraph 2. The method of paragraph 1, wherein the ROCK inhibitor is an isoquinoline or pyridine/pyrrolopyridine based ROCK inhibitors.

Paragraph 3. The method of paragraph 1, wherein the ROCK inhibitor is an indazole based ROCK inhibitor.

Paragraph 4. The method of paragraph 1, wherein the ROCK inhibitor is a pyridine, pyrrolopyridine, or pyrimidine based ROCK inhibitor.

Paragraph 5. The method of paragraph 1, wherein the ROCK inhibitor is a pyrazole based ROCK inhibitor.

Paragraph 6. The method of paragraph 1, wherein the ROCK inhibitor is a methylenephenyl substituted pyrazole based ROCK inhibitor.

Paragraph 7. The method of paragraph 1, wherein the ROCK inhibitor is a benzimidazole, benzothiazole, indole, or indazole substituted pyrazole derivatives ROCK inhibitor.

Paragraph 8. The method of paragraph 1, wherein the ROCK inhibitor is a quniazolinone substituted pyrazole derivatives ROCK inhibitor.

Paragraph 9. The method of paragraph 1, wherein the ROCK inhibitor is a thiophene substituted pyrimidine based or aminofurazane based ROCK inhibitor.

Paragraph 10. The method of paragraph 1, wherein the ROCK inhibitor is a 6- or 7-substituted isoquinoline or isoquinolinone based ROCK inhibitor.

Paragraph 11. The method of paragraph 1, wherein the ROCK inhibitor is a boron derivative ROCK inhibitor.

Paragraph 12. The method of paragraph 1, wherein the ROCK inhibitor is a soft ROCK inhibitor.

Paragraph 13. The method of paragraph 1, wherein the ROCK inhibitor is an isoform selective ROCK-II inhibitor.

Paragraph 14. The method of paragraph 1, wherein the ROCK inhibitor is Y-27632.

Paragraph 15. The method of any preceding paragraph, wherein the presence of the ROCK inhibitor throughout differentiation promotes cell viability relative to differentiation in which ROCK inhibitor contacting is not throughout differentiation.

Paragraph 16. The method of any preceding paragraph, wherein the stem cell is a pluripotent stem cell or a cardiac progenitor cell.

Paragraph 17. The method of any preceding paragraph, wherein the stem cell is human.

Paragraph 18. The method of any preceding paragraph, wherein the stem cell is an induced pluripotent stem cell.

Paragraph 19. The method of any preceding paragraph, wherein the stem cell is an embryonic stem cell.

Paragraph 20. The method of any one of paragraphs 1-19, wherein the stem cell is not an embryonic stem cell.

Paragraph 21. The method of any preceding paragraph, further comprising contacting the stem cell or a cell differentiating from the stem cell with a small molecule selected from CHIR99021 and WIKI4.

Paragraph 22. The method of any preceding claim, further comprising contacting the stem cell or a cell differentiating from the stem cell with CHIR99021 and WIKI4.

Paragraph 23. A composition comprising a stem cell, a ROCK inhibitor, and one or both of CHIR99021 and WIKI4.

Paragraph 24. The composition of paragraph 23, wherein the ROCK inhibitor is Y-27632.

Paragraph 25. The composition of paragraph 23 or 24, wherein the stem cell is a pluripotent stem cell or a cardiac progenitor cell.

Paragraph 26. The composition of any one of paragraphs 23-25, wherein the stem cell is human.

Paragraph 27. The composition of any one of paragraphs 23-26, wherein the stem cell is an induced pluripotent stem cell.

Paragraph 28. The composition of any one of paragraphs 23-27, wherein the stem cell is an embryonic stem cell.

Paragraph 29. The composition of any one of paragraphs 23-27, wherein the stem cell is not an embryonic stem cell.

Paragraph 30. A transplant composition comprising a cardiomyocyte produced by the method of any one of paragraphs 1-22.

Paragraph 31. The transplant composition of paragraph 30, further comprising a pharmaceutically acceptable carrier.

Paragraph 32. The transplant composition of paragraph 30 or 31, further comprising a gel, scaffold or matrix.

Paragraph 33. The transplant composition of paragraph 32, wherein the gel, scaffold or matrix is biodegradable.

Paragraph 34. The transplant composition of any one of paragraphs 30-33, further comprising any one or more of a solubilized basement membrane protein or preparation thereof, an immunosuppressive agent, a pan-caspase inhibitor, an anti-apoptotic agent, IGF-1, and a KATP channel opening agent.

Paragraph 35. A pharmaceutical composition comprising a cardiomyocyte produced by the method of any one of paragraphs 1-28, or a transplant composition of any one of paragraphs 30-34, in combination with a pharmaceutically acceptable carrier.

Paragraph 36. A method for improving cardiac function in a subject in need thereof, the method comprising: administering a cardiomyocyte produced by the method of any one of paragraphs 1-22, or a composition of any one of paragraphs 23-35 to cardiac tissue of the subject in need thereof.

Paragraph 37. The method of paragraph 36, wherein the subject is human.

Paragraph 38. The method of paragraph 36 or 37, wherein the subject has damaged cardiac tissue resulting from acute or chronic injury.

Paragraph 39. A cardiomyocyte produced by the method of any one of paragraphs 1-22, or a composition of any one of paragraphs 23-35 for use in a method for improving cardiac function in a subject in need thereof, said method comprising administering a cardiomyocyte produced by the method of any one of paragraphs 1-22, or a composition of any one of paragraphs 23-35 to cardiac tissue of the subject in need thereof.

Paragraph 40. The use of paragraph 39, wherein the subject is human.

Paragraph 41. The method of paragraph 39 or 40, wherein the subject has damaged cardiac tissue resulting from acute or chronic injury.

Paragraph 42. The composition of paragraph 23, wherein the contact with at least one ROCK inhibitor is initiated when CHIR99021 is added during a differentiation protocol beginning at least the first day of differentiation of the stem cell.

Paragraph 43. The composition of paragraph 23, wherein the contact with at least one ROCK inhibitor is initiated when WIKI4 is added during a differentiation protocol beginning at least the third day of differentiation of the stem cell.

### EXAMPLES

### Example 1: Differentiation of pluripotent stem cells (PSCs) to cardiomyocytes

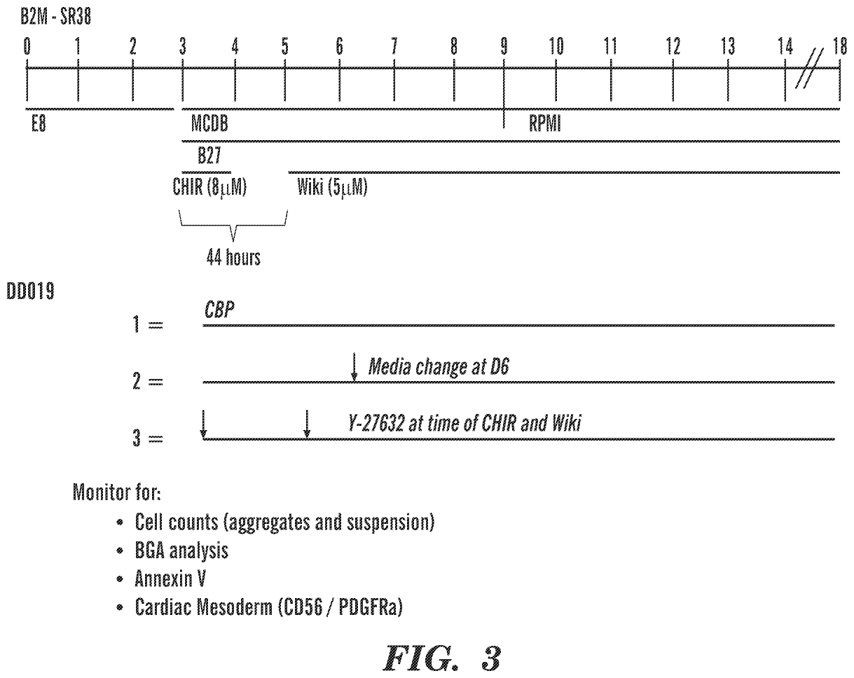

involves multiple steps of PSC expansion and differentiation to the desired lineage. As presented here PSCs are thawed from a suspension PSC bank (PMID: 26318718) and aggregated in suspension culture, with the addition of the ROCK inhibitor Y-27632, shown to permit survival of dissociated PSCs (PMID: 17529971). Following expansion, the cells are induced to differentiate to mesoderm and then cardiomyocytes by modulating Wnt signaling using CHIR99021 to induce mesoderm (PMID: 23257984), followed small molecule Wnt inhibitor, WIKI4 (PMID: 23227175) to specify mesoderm to the cardiomyocyte lineage (FIG. 3).

### Example 2: A Challenge with the Methodology Described Here is Substantial Cell

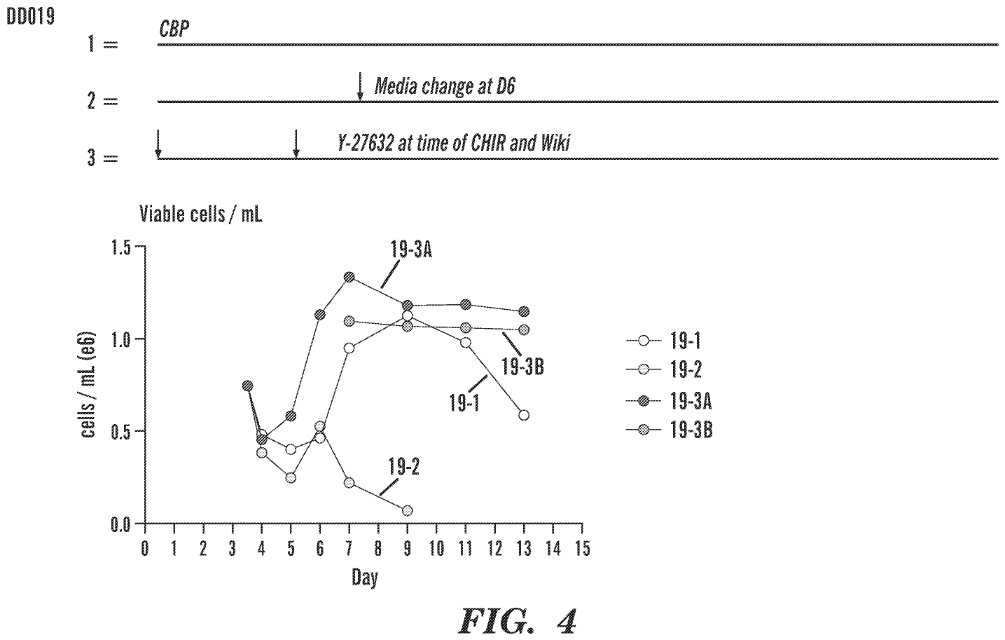

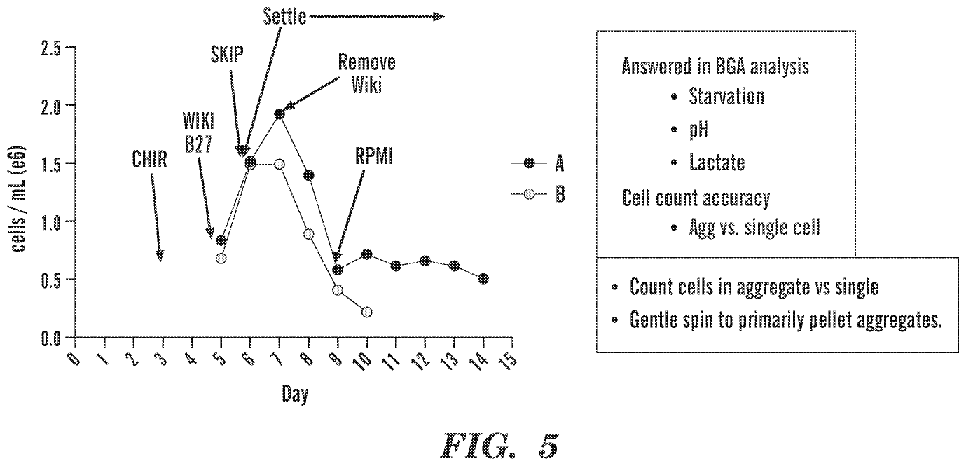

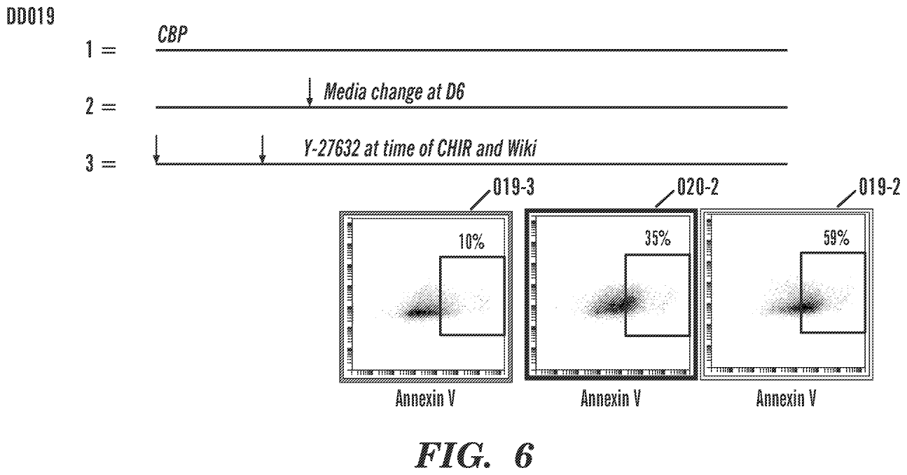

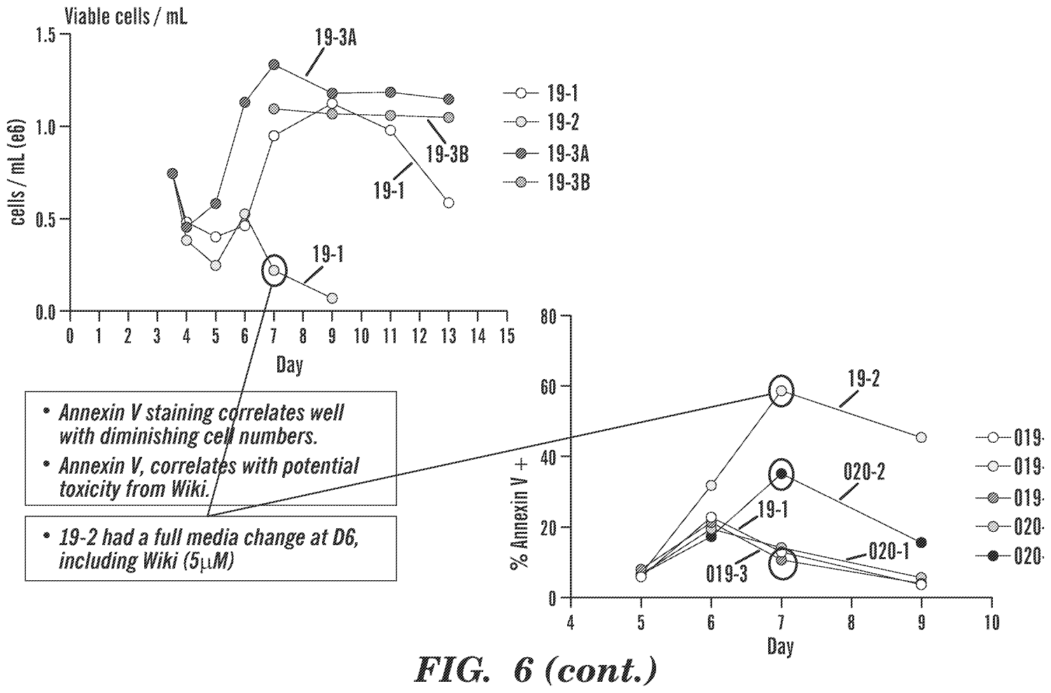

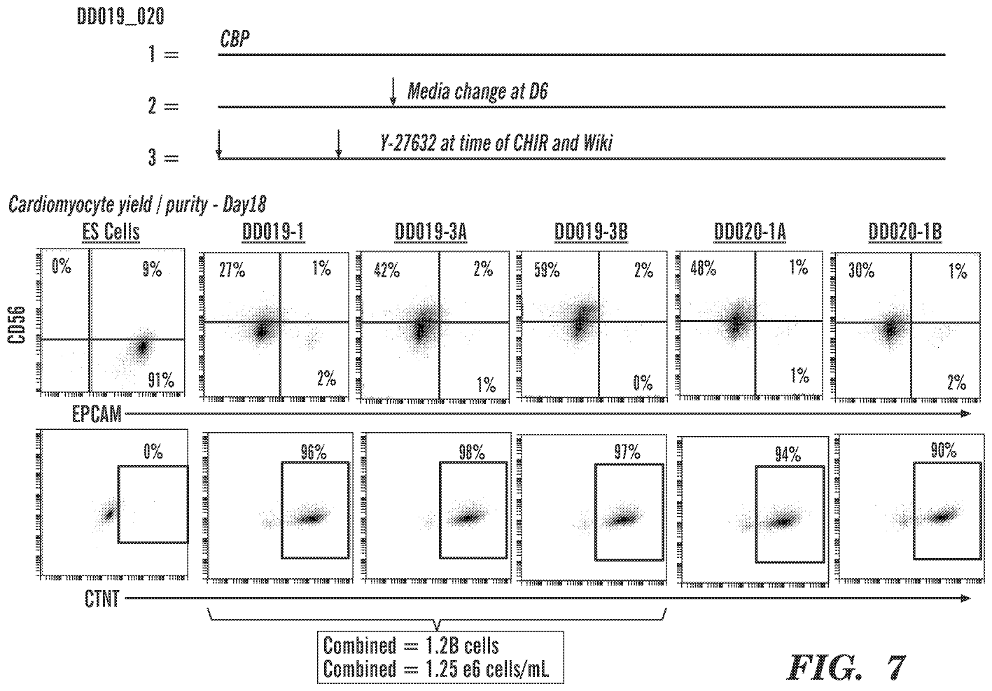

loss during the addition of CHIR99021 and WIKI4. Addition of Y-27632 at each stage of small molecule Wnt modulation dramatically improved cell yield of the differentiation cultures. For example, Y-27632 addition, in combination with CHIR99021 and WIKI4, displayed greater than double the yield of pre-cardiomyocytes when measured at day 6 of the differentiation process (1.1E6 cells/mL with Y-27632 vs. 4.7E5 cells/mL without) (FIG. 4). Additionally, at differentiation day 13, the culture with the addition of Y-27632 yielded nearly 4× more cardiomyocytes than cultures without. Subsequently Y-27632 was added to all cultures during the differentiation (FIG. 4).

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An in vitro differentiation method for preparing a cardiomyocyte from a stem cell, the method comprising contacting the stem cell with an inhibitor of Rho-associated, coiled-coil containing protein kinase (ROCK) throughout differentiation from stem cell to cardiomyocyte.

2. The method of claim 1, wherein the ROCK inhibitor is an isoquinoline or pyridine/pyrrolopyridine based ROCK inhibitor.

3. The method of claim 1, wherein the ROCK inhibitor is an indazole based ROCK inhibitor.

4. The method of claim 1, wherein the ROCK inhibitor is a pyridine, pyrrolopyridine, or pyrimidine based ROCK inhibitor.

5. The method of claim 1, wherein the ROCK inhibitor is a pyrazole based ROCK inhibitor.

6. The method of claim 1, wherein the ROCK inhibitor is a methylenephenyl substituted pyrazole based ROCK inhibitor.

7. The method of claim 1, wherein the ROCK inhibitor is a benzimidazole, benzothiazole, indole, or indazole substituted pyrazole derivative ROCK inhibitor.

8. The method of claim 1, wherein the ROCK inhibitor is a quniazolinone substituted pyrazole derivative ROCK inhibitor.

9. The method of claim 1, wherein the ROCK inhibitor is a thiophene substituted pyrimidine based or aminofurazane based ROCK inhibitor.

10. The method of claim 1, wherein the ROCK inhibitor is a 6- or 7-substituted isoquinoline or isoquinolinone based ROCK inhibitor.

11. The method of claim 1, wherein the ROCK inhibitor is a boron derivative ROCK inhibitor.

12. The method of claim 1, wherein the ROCK inhibitor is a soft ROCK inhibitor.

13. The method of claim 1, wherein the ROCK inhibitor is an isoform selective ROCK-II inhibitor.

14. The method of claim 1, wherein the ROCK inhibitor is Y-27632.

15. The method of any preceding claim, wherein the presence of the ROCK inhibitor throughout differentiation promotes cell viability relative to differentiation in which ROCK inhibitor contacting is not throughout differentiation.

16. The method of any preceding claim, wherein the stem cell is a pluripotent stem cell or a cardiac progenitor cell.

17. The method of any preceding claim, wherein the stem cell is human.

18. The method of any preceding claim, wherein the stem cell is an induced pluripotent stem cell.

19. The method of any preceding claim, wherein the stem cell is an embryonic stem cell.

20. The method of any one of claims 1-19, wherein the stem cell is not an embryonic stem cell.

21. The method of any preceding claim, further comprising contacting the stem cell or a cell differentiating from the stem cell with a small molecule selected from CHIR99021 and WIKI4.

22. The method of any preceding claim, further comprising contacting the stem cell or a cell differentiating from the stem cell with CHIR99021 and WIKI4.

23. A composition comprising a stem cell, a ROCK inhibitor, and one or both of CHIR99021 and WIKI4.

24. The composition of claim 23, wherein the ROCK inhibitor is Y-27632.

25. The composition of claim 23 or 24, wherein the stem cell is a pluripotent stem cell or a cardiac progenitor cell.

26. The composition of any one of claims 23-25, wherein the stem cell is human.

27. The composition of any one of claims 23-26, wherein the stem cell is an induced pluripotent stem cell.

28. The composition of any one of claims 23-27, wherein the stem cell is an embryonic stem cell.

29. The composition of any one of claims 23-27, wherein the stem cell is not an embryonic stem cell.

30. A transplant composition comprising a cardiomyocyte produced by the method of any one of claims 1-22.

31. The transplant composition of claim 30, further comprising a pharmaceutically acceptable carrier.

32. The transplant composition of claim 30 or 31, further comprising a gel, scaffold or matrix.

33. The transplant composition of claim 32, wherein the gel, scaffold or matrix is biodegradable.

34. The transplant composition of any one of claims 30-33, further comprising any one or more of a solubilized basement membrane protein or preparation thereof, an immunosuppressive agent, a pan-caspase inhibitor, an anti-apoptotic agent, IGF-1, and a KATP channel opening agent.

35. A pharmaceutical composition comprising a cardiomyocyte produced by the method of any one of claims 1-28, or a transplant composition of any one of claims 30-34, in combination with a pharmaceutically acceptable carrier.

36. A method for improving cardiac function in a subject in need thereof, the method comprising: administering a cardiomyocyte produced by the method of any one of claims 1-22, or a composition of any one of claims 23-35 to cardiac tissue of the subject in need thereof.

37. The method of claim 36, wherein the subject is human.

38. The method of claim 36 or 37, wherein the subject has damaged cardiac tissue resulting from acute or chronic injury.

39. A cardiomyocyte produced by the method of any one of claims 1-22, or a composition of any one of claims 23-35 for use in a method for improving cardiac function in a subject in need thereof, said method comprising administering a cardiomyocyte produced by the method of any one of claims 1-22, or a composition of any one of claims 23-35 to cardiac tissue of the subject in need thereof.

40. The use of claim 39, wherein the subject is human.

41. The method of claim 39 or 40, wherein the subject has damaged cardiac tissue resulting from acute or chronic injury.

42. The composition of claim 23, wherein the contact with at least one ROCK inhibitor is initiated when CHIR99021 is added during a differentiation protocol beginning at least the first day of differentiation of the stem cell.

43. The composition of claim 23, wherein the contact with at least one ROCK inhibitor is initiated when WIKI4 is added during a differentiation protocol beginning at least the third day of differentiation of the stem cell.
